SEQUENCE LISTING (43 sequences)

SEQ ID NO: 1 - DNA, 23 bp, Artificial Sequence
  Description of Artificial Sequence Synthetic primer
gtcctggctgctcttctacaagg

SEQ ID NO: 2 - DNA, 23 bp, Artificial Sequence
  Description of Artificial Sequence Synthetic primer
ggtacgtgctgttgaactgttcc

SEQ ID NO: 3 - DNA, 29 bp, Artificial Sequence
  Description of Artificial Sequence Synthetic primer
gatgtgcagctgcaggagtctggrggagg

SEQ ID NO: 4 - DNA, 35 bp, Artificial Sequence
  Description of Artificial Sequence Synthetic primer
ggactagtgcggccgctggagacggtgacctgggt

SEQ ID NO: 5 - protein, 144 aa, Lama glama
QVQLQESGGGLVQPGGSLRLSCRASGFTFANYDLAWTRQVPGKEREFVASIGVARGRTRTPATFYSASAKGRFTVSRDDARNTVYLQMNSLKPEDSAVYYCAAKDVEVSVATVEDYPYWGRGTQVTVSSGRYPYDVPDYGSGRA

SEQ ID NO: 6 - protein, 139 aa, Lama glama
QVQLQESGGGLVQPGGSLRLSCAASGLTFANYDLAWSRQAPGKQREFVASIGVTRNPPYYSGSVKGRFTVSRDNAKETVYLQMNDLKPEDSAVYYCAAKDASVTVATIEDYPYWGRGTQVTVSSGRYPYDVPDYGSGRA

SEQ ID NO: 7 - protein, 136 aa, Lama glama
QVQLQESGGGSVQAGGSLRLSCVASGRTFSSYAMAWFRQVPGKEREFVGRISRSGGSTMYADSVKGRFDISRDNAKNTVFLQMSSLKPEDTAVYYCAAKAAVILTGIPDYWGQGTQVTVSSGRYPYDVPDYGSGRA

SEQ ID NO: 8 - protein, 144 aa, Lama glama
QVQLQESGGGLVQPGGSLKLSCRASGFTFANYDLAWTRQVPGKEREFVASIGVARGRTRTPATFYSASVKGRFTVSRDDARNTVYLQMNSLKPEDSAVYYCAAKDVEVSVATVEDYPYWGRGTQVTVSSGRYPYDVPDYGSGRA

SEQ ID NO: 9 - protein, 134 aa, Lama glama
QVQLQESGGGLVQAGTSLVLSCAISGHTFSITGTAWFRQAPGKEREFVAGLTSSGDITHYASSVKGRFTISSDIAKNTVYLQMNNLKPEDTAVYYCTSVIRGRAGYDTWGQGTQVTVSSGRYPYDVPDYGSGRA

SEQ ID NO: 10 - protein, 136 aa, Lama glama
QVQLQESGGGLVQAGGSLRLSCVASGRTFSSYAMAWFRQVPGKEREFVGRISRSGGSTMYADSVKGRFDISRDNAKNTVFLQMSSLKPEDTAVYYCAAKAAVILTGIPDYWGQGTQVTVSSGRYPYDVPDYGSGRA

SEQ ID NO: 11 - protein, 136 aa, Lama glama; identical to SEQ ID NO: 7

SEQ ID NO: 12 - protein, 136 aa, Lama glama; identical to SEQ ID NO: 7

SEQ ID NO: 13 - protein, 134 aa, Lama glama; identical to SEQ ID NO: 9

SEQ ID NO: 14 - protein, 136 aa, Lama glama; identical to SEQ ID NO: 7

SEQ ID NO: 15 - protein, 136 aa, Lama glama; identical to SEQ ID NO: 7

SEQ ID NO: 16 - protein, 139 aa, Lama glama
QVQLQESGGGLVQPGGSQRLSCAASGLTFANYDLAWSRQAPGKQREFVASIGVTRNPPYYSGSVKGRFTVSRDNAKETVYLQMNDLKPEDSAVYYCAAKDASVTVATIEDYPYWGRGTQVTVSSGRYPYDVPDYGSGRA

SEQ ID NO: 17 - protein, 139 aa, Lama glama
QVQLQESGGGLVQTGGSLRLSCKASGRAFARYDLAWSRQAPGKQREFVASIGVTRNPPYYSGSVKGRFTVSRDNAKETVYLQMNDLKPEDSAVYYCAAKDASVTVATIEDYPYWGRGTQVTVSSGRYPYDVPDYGSGRA

SEQ ID NO: 18 - protein, 136 aa, Lama glama; identical to SEQ ID NO: 10

SEQ ID NO: 19 - protein, 9 aa, Lama glama
FTFANYDLA

SEQ ID NO: 20 - protein, 13 aa, Lama glama
SIGVARGRTRTPA

SEQ ID NO: 21 - protein, 15 aa, Lama glama
KDVEVSVATVEDYPY

SEQ ID NO: 22 - protein, 9 aa, Lama glama
LTFANYDLA

SEQ ID NO: 23 - protein, 9 aa, Lama glama
RAFARYDLA

SEQ ID NO: 24 - protein, 8 aa, Lama glama
SIGVTRNP

SEQ ID NO: 25 - protein, 15 aa, Lama glama
KDASVTVATIEDYPY

SEQ ID NO: 26 - protein, 9 aa, Lama glama
HTFSITGTA

SEQ ID NO: 27 - protein, 8 aa, Lama glama
GLTSSGDI

SEQ ID NO: 28 - protein, 10 aa, Lama glama
VIRGRAGYDT

SEQ ID NO: 29 - protein, 9 aa, Lama glama
RTFSSYAMA

SEQ ID NO: 30 - protein, 8 aa, Lama glama
RISRSGGS

SEQ ID NO: 31 - protein, 12 aa, Lama glama
KAAVILTGIPDY

SEQ ID NO: 32 - protein, 128 aa, Lama glama
QVQLQESGGGLVQTGGSLRLSCKASGRAFARYDLAWSRQAPGKQREFVASIGVTRNPPYYSGSVKGRFTVSRDNAKETVYLQMNDLKPEDSAVYYCAAKDASVTVATIEDYPYWGRGTQVTVSSGRYP

SEQ ID NO: 33 - protein, 123 aa, Lama glama
QVQLQESGGGLVQAGTSLVLSCAISGHTFSITGTAWFRQAPGKEREFVAGLTSSGDITHYASSVKGRFTISSDIAKNTVYLQMNNLKPEDTAVYYCTSVIRGRAGYDTWGQGTQVTVSSGRYP

SEQ ID NO: 34 - protein, 133 aa, Lama glama
QVQLQESGGGLVQPGGSLRLSCRASGFTFANYDLAWTRQVPGKEREFVASIGVARGRTRTPATFYSASAKGRFTVSRDDARNTVYLQMNSLKPEDSAVYYCAAKDVEVSVATVEDYPYWGRGTQVTVSSGRYP

SEQ ID NO: 35 - protein, 128 aa, Lama glama; identical to SEQ ID NO: 32

SEQ ID NO: 36 - protein, 127 aa, Lama glama
QVQLVESGGGLVQAGGSLRLSCTASGRTFSDYDMAWFRQAPGKERDRVSAISTKGGSTWYHDSVKGRFTISRDNAKNTVYLQMNSLKPEDTAVYYCAAGAVTYYSARYEYDYWGQGTQVTVSSHHHH

SEQ ID NO: 37 - protein, 122 aa, Lama glama
QVQLQESGGGLVQAGGSLRLSCAVSGRTFSNYYSGWFRQAPGKEREFLASIRWSDSTINYASDSVKGRFTISRDTAKNTVYLQMNSLKLEDTAVYHCAARRLATYDYWGQGTQVTVSSGRYP

SEQ ID NO: 38 - protein, 128 aa, Lama glama
QLVESGGGLVQAGGSLRVSCAASGRTFSSYSMGWFRQAPGKEREFVAAISRSDNSTYYADSVKGRFTISRDSAKNTVYLQMNSLKPEDTAVYYCAATPYGSRYYLRELREYDYWGQGTQVTVSSHHHH

SEQ ID NO: 39 - protein, 238 aa, Homo sapiens
ENFNVATEESSEPLGEDDFEMFYETWEKFDPDATQFIAYSRLSDFVDTLQEPLRIAKPNKIKLITLDLPMVPGDKIHCLDILFALTKEVLGDSGEMDALKQTMEEKFMAANPSKVSYEPITTTLKRKHEEVCAIKIQRAYRRHLLQRSMKQASYMYRHSHDGSGDDAPEKEGLLANTMSKMYGHENGNSSSPSPEEKGEAGDAGPTMGLMPISPSDTAWPPAPPPGQTVRPGVKESLV

SEQ ID NO: 40 - protein, 244 aa, Homo sapiens
ENFSVATEESTEPLSEDDFDMFYEIWEKFDPEATQFIEYSVLSDFADALSEPLRIAKPNQISLINMDLPMVSGDRIHCMDILFAFTKRVLGESGEMDALKIQMEEKFMAANPSKISYEPITTTLRRKHEEVSAMVIQRAFRRHLLQRSLKHASFLFRQQAGSGLSEEDAPEREGLIAYVMSENFSRPLGPPSSSSISSTSFPPSYDSVTRATSDNLQVRGSDYSHSEDLADFPPSPDRDRESIV

SEQ ID NO: 41 - protein, 228 aa, Homo sapiens
ENFSVATEESTEPLSEDDFEMFYEVWEKFDPDATQFIEFSKLSDFAAALDPPLLIAKPNKVQLIAMDLPMVSGDRIHCLDILFAFTKRVLGESGEMDSLRSQMEERFMSANPSKVSYEPITTTLKRKQEDVSATVIQRAYRRYRLRQNVKNISSIYIKDGDRDDDLLNKKDMAFDNVNENSSPEKTDATSSTTSPPSYDSVTKPDKEKYEQDRTEKEDKGKDSKESKK

SEQ ID NO: 42 - protein, 186 aa, Homo sapiens
ENFNTATEESEDPLGEDDFDIFYEVWEKFDPEATQFIKYSALSDFADALPEPLRVAKPNKYQFLVMDLPMVSEDRLHCMDILFAFTARVLGGSDGLDSMKAMMEEKFMEANPLKKLYEPIVTTTKRKEEERGAIIQKAFRKYMMKVTKGDQGDQNDLENGPHSPLQTLCNGDLSSFGVAKGKVHCD

SEQ ID NO: 43 - protein, 6 aa, Artificial Sequence
  Description of Artificial Sequence Synthetic 6xHis tag
HHHHHH

(19) United States
(12) Patent Application Publication
(10) Pub. No.: US 2023/0406919 A1
(43) Pub. Date: Dec. 21, 2023

(54) NANOBODIES WITH SPECIFIC AFFINITY FOR VOLTAGE GATED SODIUM CHANNELS

(71) Applicants: The Johns Hopkins University, Baltimore, MD (US); Fundacion Instituto Lelior, Buenos Aires (AR); Consejo Nacional de Investigacion Cientifica y Tecnica (CONICET), Caba (AR)

(72) Inventors: Sandra B. Gabelli, Baltimore, MD (US); Leon M. Amzel, Baltimore, MD (US); Lakshmi Srinivasan, Baltimore, MD (US); Vanina Alzogaray, Buenos Aires (AR); Manu Ben-Johny, Baltimore, MD (US)

(21) Appl. No.: 18/037,531

(22) PCT Filed: Dec. 3, 2021
(86) PCT No.: PCT/US2021/061874

§ 371 Date: May 17, 2023

Related U.S. Application Data
(60) Provisional application No. 63/121,078, filed on Dec. 3, 2020.

(51) Int. Cl. C07K 16/28; A61K 48/00; G01N 33/68; G01N 33/542

(52) U.S. Cl. CPC C07K 16/28; A61K 48/0041; G01N 33/6893; G01N 33/542; C07K 2317/569; C07K 2317/22; C07K 2317/24

(57) ABSTRACT

Provided herein a single-domain antibodies (sdAbs) that bind to voltage-gated sodium channel (Naᵥ)1.4 or Naᵥ1.5 and polynucleotides encoding the sdAb. Also provided herein are expression cassettes, vectors and host cells including polynucleotides N encoding the sdAb, and pharmaceutical composition including the sdAb, and methods of use thereof. The methods include the detection and/or capture Naᵥ1.4 or Naᵥ1.5 in a sample, the detection of a disease or condition in a subject, and the treatment of cardiac arrhythmia, myotonia, sudden cardiac death syndrome or cancer in a subject using sdAbs that bind Naᵥ 1.4 or Naᵥ 1.5.

### CROSS-REFERENCE TO RELATED APPLICATIONS

This application claims priority under § 119(e) to U.S. Provisional Application Ser. No. 63/121,078, filed Dec. 3, 2020, which is hereby incorporated by reference in its entirety.

### STATEMENT REGARDING FEDERALLY SPONSORED RESEARCH OR DEVELOPMENT

This invention was made with government support under grant HL128743 awarded by the National Institutes of Health. The government has certain rights in the invention

### BACKGROUND OF THE INVENTION

### Field of the Invention

The present invention relates generally to binding agents and more specifically to nanobodies having binding affinity for voltage gated sodium channels.

### Background Information

The nine human isoforms of voltage-gated sodium channels (Naᵥ1.1-1.9) rapidly respond to changes in cellular membrane potential by allowing Na′ ions to move into the cell. They play an important role in the generation of the action potential in excitable tissues such as skeletal muscle, heart and nerves. The nine (9) human voltage-gated sodium channel isoforms have unique functions, wide cell and tissue distribution and implications in human genetic diseases such as cardiac arrhythmias, myotonias and neuropathic pain. Mutations in the C-terminal cytoplasmic region of these proteins have been implicated in human genetic diseases such as hypokalemic periodic paralysis, myotonia, Long QT syndrome and Brugada syndrome. Despite the physiological importance of the Naᵥ isoforms in normal physiology and disease, it has been challenging to target each of them with specificity owing to their high sequence identity. To achieve tissue specificity and to avoid off-target side effects of anti-Nay antibodies, there is an increasing need for biologicals with high solubility, stability and specificity.

Nanobodies (Nbs) are single, variable heavy chain (VHH) immunoglobulin (Ig) domains derived by antigen stimulation of camelids such as camels, llamas and alpacas. Following immunization, the camelids produce, among the normal Ig response, special heavy-chain only antibodies (hcAb) harboring the VHH. Nbs, single Ig domain proteins, are small prolate-shaped molecules (<15 kDa) that retain the epitope-recognizing function of an antibody. Nbs may be selected to contain an extended and more flexible CDR3 loop than the regular VH domains, partly contributing to their high epitope affinity and their ability to better access smaller and cryptic epitopes. Moreover, VHH domains are amenable to cloning and protein modifications, and can be produced in bacterial expression systems in scalable amounts. Nbs also display superior solubility, stability, in vivo half-lives and pharmacodynamics compared to conventional antibodies. For example, Nbs to P2x channel proteins have been shown to display greater therapeutic potential than antibodies by modulating channel function, and reducing the in vivo inflammation caused by P2X7. Nbs have also been used as crystallization chaperones, visualization agents, in vivo radiotracers, pulldown baits, intracellular pathways modulators, virus neutralization agents, and therapeutics agents.

Currently, there are no Naᵥ1.5 or Naᵥ1.4 channel specific antibodies or binding agents that recognize the full protein and do not cross react with other voltage gated sodium channels. Currently available antibodies against Naᵥs were raised against only short peptides, 10-20 aa, and are used as molecular probes. There is an unmet need for specific nanobodies that recognize and bind to certain voltage gated sodium channels without cross-reactivity to other isoforms.

### SUMMARY OF THE INVENTION

The present invention is based on the seminal discovery of llama-derived single-domain antibodies having binding affinity specificity toward voltage-gated sodium channel (Naᵥ)1.4 or Naᵥ1.5.

In one embodiment, the invention provides a single-domain antibody (sdAb) that specifically binds to voltage-gated sodium channel (Naᵥ)1.4 or Naᵥ1.5.

In one aspect, the sdAb is selected from a camelid sdAb or a humanized sdAb. In one aspect, the sdAb is a llama sdAb or a humanized llama sdAb. In one aspect, the sdAb includes a complementarity-determining region (CDR) 1 having an amino acid sequence as set forth in SEQ ID NO:19, 22, 23, 26 or 29; a CDR2 having an amino acid sequence as set forth in SEQ ID NO:20, 24, 27 or 30; and a CDR3 having an amino acid sequence as set forth in SEQ ID NO:21, 25, 28 or 31. In some aspects, the sdAb has a CDR1 having an amino acid sequence as set forth in SEQ ID NO:19, a CDR2 having an amino acid sequence as set forth in SEQ ID NO:20, and a CDR3 having an amino acid sequence as set forth in SEQ ID NO:21. In other aspects, the sdAb has a CDR1 having an amino acid sequence as set forth in SEQ ID NO:23, a CDR2 having an amino acid sequence as set forth in SEQ ID NO:24, and a CDR3 having an amino acid sequence as set forth in SEQ ID NO:25. In one aspect, the amino acid sequence of the sdAb is set forth in SEQ ID NO:5 or 17. In one aspect, the sdAb does not bind to Naᵥ1.7 or Naᵥ1.9. In other aspects, the sdAb binds to Naᵥ1.4 or Naᵥ1.5 with a nanomolar affinity.

In another embodiment, the invention provides an isolated polynucleotide encoding a sdAb that specifically binds to Naᵥ1.4 or Naᵥ1.5.

In one aspect, the polynucleotide has an amino acid sequence as set forth in any of SEQ ID NOs:5-18. In other aspects, the polynucleotide has an amino acid sequence as set forth in SEQ ID NO:5 or 17.

In one embodiment, the invention provides an expression cassette including an isolated polynucleotide encoding a sdAb that specifically binds to Naᵥ1.4 or Naᵥ1.5. In one aspect, the expression cassette further includes a polynucleotide encoding a protein tag. In another aspect, the polynucleotide encoding the sdAb is operably linked to the polynucleotide encoding the protein tag to encode a fusion protein.

In another embodiment, the invention provides a vector including an expression cassette including an isolated polynucleotide encoding a sdAb that specifically binds to Naᵥ1.4 or Naᵥ1.5.

In an additional embodiment, the invention provides a host cell including a polynucleotide encoding a sdAb that specifically binds to Naᵥ1.4 or Naᵥ1.5, expression cassette including a polynucleotide encoding a sdAb that specifically binds to volNaᵥ1.4 or Naᵥ1.5, or a vector including an expression cassette including an isolated polynucleotide encoding a sdAb that specifically binds to Naᵥ1.4 or Naᵥ1.5.

In one embodiment, the invention provides a pharmaceutical composition including the sdAb described herein and a pharmaceutically acceptable carrier.

In another embodiment, the invention provides a method of detecting and/or capturing Naᵥ1.4 or Naᵥ1.5 in a sample including contacting the sample with the sdAb described herein; and detecting and/or capturing a complex between the sdAb and the Naᵥ1.4 or Naᵥ1.5.

In one aspect, detecting the complex is by western blot, immunohistochemistry, flow cytometry, ELISA or immunofluorescence. In other aspects, capturing the complex is by immunoprecipitation (IP) or co-IP. In one aspect, the sample is a tissue or cell derived from a cardiac tissue, a skeletal muscle tissue, a nerve tissue or a lysate thereof. In other aspects, the sample is from a tissue or cell from a subject who has cancer.

In one embodiment, the invention provides a method of detecting a disease or condition in a subject including contacting a sample from the subject with the sdAb described herein and detecting the sdAb in the sample.

In one aspect, the disease or condition is selected from the group consisting of cardiac arrhythmia, myotonia, neuropathic pain, hypokalemic periodic paralysis, Long QT syndrome, sudden cardiac death syndrome and Brugada syndrome. In other aspects, the disease or condition is selected from the group consisting of colon, prostate, breast, cervical, lung, pancreas, biliary, rectal, liver, kidney, testicular, brain, head and neck, ovarian cancer, melanoma, sarcoma, multiple myeloma, leukemia, and lymphoma.

In another embodiment, the invention provides a method of treating cardiac arrhythmia, myotonia or sudden cardiac death syndrome in a subject including administering to the subject a single-domain antibody (sdAb) that specifically binds to voltage-gated sodium channel (Naᵥ)1.4 or Naᵥ1.5 for tissue-specific targeting of Naᵥ1.4 or Naᵥ1.5.

In one aspect, the sdAb has an amino acid sequence as set forth in SEQ ID NO:5 or 17.

In an additional embodiment, the invention provides a method of treating cancer in a subject including administering to the subject a single-domain antibody (sdAb) that specifically binds to voltage-gated sodium channel (Naᵥ)1.4 or Naᵥ1.5 for tissue-specific targeting of Naᵥ1.4 or Naᵥ1.5.

In one aspect, the cancer is selected from the group consisting of colon, prostate, breast, cervical, lung, pancreas, biliary, rectal, liver, kidney, testicular, brain, head and neck, ovarian cancer, melanoma, sarcoma, multiple myeloma, leukemia, and lymphoma. In another aspect, the sdAb has an amino acid sequence as set forth in SEQ ID NO:5 or 17.

### BRIEF DESCRIPTION OF THE DRAWINGS

FIGS. 1A-1B show the serum total IgG quantification by ELISA. FIG. 1A illustrates the results of an ELISA of immune sera from llama at day 35 following immunization with CTNaᵥ1.4T-CaM (aa 1594-1764). The graphed absorbance was subtracted from the control value without antigen depending on the dilution of serum from llama #1. The symbols correspond to the sera titer obtained for antigen CTNaᵥ1.4T-CaM. Black circles indicate pre-immune sera from Day 0 and black squares the sera from Day 35. FIG. 1B is a sequence alignment of 14 different llama derived anti-CTNaᵥ1.4 Nbs classified into 4 unique Nb families (1-4) selected for heterologous protein expression in E. coli.

FIGS. 2A-2E show the design and expression of anti-Naᵥ1.4 specific Nbs. FIG. 2A is a graph illustrating phage ELISA of 14 different anti-Naᵥ1.4 Nb classes using CTNaᵥ1.4T-CaM as bait. The ‘+’ sign indicates the selected Nb clones for expression. Absorbances higher than 1 (dashed line) were considered positive. FIG. 2B is a graph illustrating the result of ELISA of the periplasmic extract using selected clones from A. Absorbances higher than 2 (dashed line) were considered positive. FIG. 2C shows an SDS-PAGE gel of IMAC purification of Nb17; FIG. 2D shows an SDS-PAGE gel of IMAC purification of Nb82. M: Molecular Weight marker; S: supernatant; FT: flow-through, W: wash, E: elution fractions. FIG. 2E is a size exclusion chromatogram of Nb17 and Nb82.

FIGS. 3A-3F show crystal structure of Nb82. FIG. 3A is a cartoon representation of Nb82 displaying the CDR1, CDR2 and CDR3. FIG. 3B is a cartoon representation of Nb82 with 180° rotation along the vertical axis. FIG. 3C is a Bird's eye view of Nb82 with surface shading according to the CDRs. FIG. 3D. is a Bird's eye view of Nb82 with surface shaded according to the electrostatic charges. FIG. 3E is a Bird's eye view of Nb82 as in FIG. 3C with ribbon representation with the CDR and residues as sticks. FIG. 3F shows sequence alignment of llama derived anti-CTNav1.4 Nbs selected for heterologous protein expression in E. coli. Identical amino acids are in white with light background, similar amino acids are in grey with white background, and different amino acids are in black. The secondary structure elements of Nb82 is placed on top of the alignment.

FIGS. 4A-4B show that the structural alignment of llama Nbs highlights the diversity in the CDR3 fold. FIG. 4A illustrates the structure of Nbs displaying the CDR3 regions (boxed) of Nb82, PDB IDs 5LMJ, 6H6H and 5LZ0. FIG. 4B is a sequence alignment of Nb82, Nb30, Nb17 and other Nbs shown in FIG. 4A displaying divergent CDR regions, CDR1, CDR 2, and CDR3 residues. Identical amino acids are in white with light background, similar amino acids are in grey with white background, different amino acids are in black with white background.

FIGS. 5A-5B show that Nb17 and Nb82 specifically recognize the Nay-muscle isoforms. FIG. 5A is ELISA bar graphs of purified Nb17, absence of Nb17, Nb82, and absence of Nb82. The dark box clusters Naᵥ proteins that represent muscle isoforms CTNaᵥ1.4T-CaM, CTNaᵥ1.4FL-CaM, CTNaᵥ1.5T-CaM, and CTNaᵥ1.5FL-CaM. The light box clusters the other Naᵥ isoforms tested, CTNaᵥ1.7T-CaM, CTNaᵥ1.7FL-CaM, CTNaᵥ1.9T, CTNaᵥ1.9FL, and CaM. FIG. 5B shows sequence alignment of CTNaᵥ1.4FL, CTNaᵥ1.5FL, CTNaᵥ1.7FL, and CTNaᵥ1.9FL used in A. The shading code is similar as in FIG. 3F.

FIG. 6 shows an ELISA plate illustrating the specificity of Nb17 and Nb82 to CTNaᵥ-muscle isoforms.

FIGS. 7A-7H show that Nb82 forms a stable complex with CTNaᵥ1.4-CaM and CTNaᵥ1.5-CaM. FIG. 7A is a size exclusion chromatography profile for CTNaᵥ1.4T-CaM+Nb82 (solid line) showing the appearance of the peak of the complex to the left compared with CTNaᵥ1.4T-CaM (dashed line). FIG. 7B is a size exclusion chromatography profile for CTNaᵥ1.4FL-CaM. FIG. 7C is an SDS-PAGE of the elution fractions from FIG. 7A. FIG. 7D is an SDS-PAGE of the elution fractions from FIG. 7B. FIG. 7E is a size exclusion chromatography profile for CTNaᵥ1.5T-CaM+Nb82 (solid line) showing the appearance of the peak of the complex to the left compared with CTNaᵥ1.5T-CaM (dashed line). FIG. 7F is a size exclusion chromatography profile for CTNaᵥ1.5FL-CaM+Nb82. FIG. 7G is an SDS-PAGE of the elution fractions from FIG. 7E. FIG. 7H is an SDS-PAGE of the elution fractions from FIG. 7F.

FIGS. 8A-8D show that Nb17 forms a stable complex with CTNaᵥ1.5-CaM.

FIG. 8A is a size exclusion chromatography profile for CTNaᵥ1.5T-CaM+Nb17 (solid line) compared with CTNaᵥ1.5T-CaM (dashed line). The Nb17 elution profile is shown in dark line. FIG. 8B is a size exclusion chromatography profile for CTNaᵥ1.5FL-CaM+Nb17 (solid line, 2 peaks) compared with CTNaᵥ1.5FL-CaM (dashed line). The Nb17 elution profile is shown in dark line. FIG. 8C is an SDS-PAGE of the fractions from FIG. 8A. FIG. 8D is an SDS-PAGE of the fractions from FIG. 8B.

FIG. 9A-9D show that Nb17 binds to CTNaᵥ1.4T-CaM and CTNaᵥ1.5T-CaM with nanomolar affinity and thermally stabilize CTNaᵥ1.4. FIG. 9A is a BLI sensorgram of Nb17 titrated with CTNaᵥ1.4T-CaM at concentrations 0.25, 12.5, 25, 50, 100, 200 nM plotted as RU in nm with time in seconds along the X-axis. FIG. 9B is a melting curve of CTNaᵥ1.4-CaM+Nb17 displaying increased thermo-stability of the CTNaᵥ1.4T-CaM (light) and CTNaᵥ1.4FL-CaM (dark) complexes by 17° C. FIG. 9C is the same sensorgram as in FIG. 9A but for CTNaᵥ1.5T-CaM. FIG. 9D is a sensorgram showing the absence of binding of Nb17 to CaM.

FIGS. 10A-10B shows BLI response curves showing no binding of Nb17 to CTNaᵥ1.7T-CaM and CTNaᵥ1.9T. FIG. 10A is a BLI sensorgram of CTNaᵥ1.7T-CaM at concentrations 0.25, 12.5, 25, 50, 100 and 200 nM titrated with Nb17. FIG. 10B is the same BLI sensorgram as in FIG. 10A but with CTNaᵥ1.9T and Nb17. Sensorgrams show no binding of Nb17 to non-muscle CTNaᵥ-CaM isoform proteins.

FIGS. 11A-11D show that Nb82 binds to CTNaᵥ1.4T-CaM and CTNaᵥ1.5T-CaM with nanomolar affinity and thermally stabilize CTNaᵥ1.4. FIG. 11A is a BLI sensorgram of Nb Nb82 17 titrated with CTNaᵥ1.4T-CaM at concentrations 0.25, 12.5, 25, 50, 100, 200 nM plotted as RU in nm with time in seconds along the X-axis. FIG. 11B is a melting curve of CTNaᵥ1.4-CaM+Nb82 displaying increased thermo-stability of the CTNaᵥ1.4T-CaM (light) and CTNaᵥ1.4FL-CaM (dark) complexes by 17° C. FIG. 11C is the same sensorgram as in FIG. 9A but for CTNaᵥ1.5T-CaM. FIG. 11D is a sensorgram showing the absence of binding of Nb82 to CaM.

FIGS. 12A-12B show BLI response curves showing No-binding of Nb82 to CTNaᵥ1.7T-CaM and CTNaᵥ1.9T. FIG. 12A is a BLI sensorgram of CTNaᵥ1.7T-CaM at concentrations 0.25, 12.5, 25, 50, 100 and 200 nM titrated with Nb82. FIG. 12B is the same BLI sensorgram as in FIG. 12A but with CTNaᵥ1.9T and Nb82. Sensorgrams show no binding of Nb82 to non-muscle CTNaᵥ-CaM isoform proteins.

FIGS. 13A-C show Western blots showing Nb82 detects nav1.4 and nav1.5 from tissues. FIG. 13A is a Western blot showing Nb82-His used as the primary antibody recognizing Naᵥ1.4(5) channels from tissues; mouse heart, mouse skeletal muscle and Naᵥ1.5 from HEK293 and hiPSC-CMs, developed using an anti-His-HRP-antibody. FIG. 13B is the same western blot as in FIG. 13A developed using a Pan-Nav antibody (Sigma). FIG. 13C is a Western blot of purified CTNav-CaM proteins, CaM alone and Nb17-His, Nb82-His and CaM alone showing positive signals for CTNaᵥ1.4T/FL, CTNaᵥ1.5T/FL and Nb17-His, Nb82-His and Nb82-StrepII developed using Nb82-His as primary antibody and anti-HisHRP antibody as secondary. All western blotting data shows one representative experiment of three.

FIGS. 14A-14D show nanobodies as tools to detect Naᵥ channels from tissue homogenates and live cells. FIG. 14A shows schematic of FRET 2-hybrid assay to probe live-cell binding of Nbs to holo-Naᵥ1.5 channels. Nbs tethered to cerulean serve as a FRET donor, while versus attached to Naᵥ1.5 serves as a FRET acceptor. FIG. 14B shows robust FRET is observed between Nb17 and Naᵥ1.5. FRET efficiency (E_{A}) is plotted against the free donor concentration (D_{free}). Each cell represents data from a single cell. FIG. 14C shows analysis of Nb17 also shows strong FRET with Naᵥ1.5. FIG. 14D shows no appreciable FRET is observed between Naᵥ1.5-venus and cerulean alone.

FIGS. 15A-15B show nanobody thermal stability and crystal structure of Nb82. FIG. 15A shows DSC curve showing the temperature denaturation of Nb17 undergoing reversible denaturation with T_{M} centered at 75.8° C. FIG. 15B shows DSC curve showing the temperature denaturation of Nb82 undergoes irreversible denaturation with T_{M} centered at 66.0° C.

FIGS. 16A-16I show effect of nanobody on Naᵥ1.5 wild-type biophysical properties. FIG. 16A shows in top panel exemplar current recordings for wild-type Naᵥ1.5 channels elicited in response to a family of voltage steps to −60 to +50 mV from a holding potential of −120 mV and in bottom panel show population data shows average peak current density (Jₚₑₐₖ)—voltage relationship. Each dot, mean±s.e.m with n denoted in parenthesis. FIG. 16B and FIG. 16C show overexpression of both Nb17 and Nb82, respectively, does not appreciably alter peak current density compared to that measured at baseline (control, FIG. 16A). Formatted as in FIG. 16A. FIG. 16D shows steady-state inactivation curve (h∞ curve) elicited using step-depolarization from a holding potential of −120 mV. Each dot, mean±s.e.m with n denoted in parenthesis. FIG. 16E shows overexpression of Nb17 lead to an 8 mV hyperpolarized shift in h∞ curve (control, FIG. 16D). FIG. 16F shows overexpression Nb82 lead to an 6 mV hyperpolarized shift h∞ curve (control, FIG. 16D). FIG. 16G shows population data shows recovery from activation (f_{recovered}) elicited using step-depolarizations from a holding potential of −120 mV at different times intervals. Each dot, mean±s.e.m with n denoted in parenthesis. FIG. 16H and FIG. 16I shows overexpression of both Nb17 and Nb82, respectively, does not appreciably alter recovery from activation (control, FIG. 16G). Format as in panel FIG. 16G.

FIGS. 17A-17B shows Transfections of a NanoMaN fusion protein (Nanobody-HectDomainE3ligase) reduces the sodium current (Ina) compared to control. FIG. 17A shows in top panel exemplar current recording for Naᵥ1.5 wild-type GFP control channels elicited in response to a family of voltage steps to −60 to +50 mV from a holding potential of −120 mV and in bottom panel show population data shows average peak current density (Jₚₑₐₖ)—voltage relationship. Each dot, mean±s.e.m with n denoted in parenthesis. FIG. 17B shows in top panel exemplar current recording for Naᵥ1.5 wild-type+nanoMaN Nb17 channels elicited in response to a family of voltage steps to −60 to +50 mV from a holding potential of −120 mV and in bottom panel show population data shows average peak current density (Jₚₑₐₖ)—voltage relationship. Each dot, mean±s.e.m with n denoted in parenthesis.

FIGS. 18A-18B shows systematic analysis of nanobody interaction with Naᵥ CTDs. FIG. 18A shows Flow-cytometric FRET 2-hybrid analysis reveals variable interaction of Nb17 with Naᵥ channels. Each dot, FRET efficiency (E_{A}) calculated from an individual cell. Top, Naᵥ1.2 binds Nb17 with a weak affinity. Middle, Naᵥ1.4 binds strongly to Nb17. Bottom, Naᵥ1.8 exhibits minimal binding to Nb17. FIG. 18B shows bar graph summary of relative association constants (K_{a,eff}) deduced from Flow-cytometric FRET 2-hybrid assay with a 1:1 binding model.

### DETAILED DESCRIPTION OF THE INVENTION

The present invention is based on the seminal discovery of llama-derived single-domain antibodies having affinity, selectivity and specificity toward voltage-gated sodium channel (Naᵥ)1.4 or Naᵥ1.5.

Before the present compositions and methods are described, it is to be understood that this invention is not limited to particular compositions, methods, and experimental conditions described, as such compositions, methods, and conditions may vary. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, it will be understood that modifications and variations are encompassed within the spirit and scope of the instant disclosure. Illustrative methods and materials are now described.

In one embodiment, the invention provides a single-domain antibody (sdAb) that specifically binds to voltage-gated sodium channel (Naᵥ)1.4 or Naᵥ1.5.

Voltage-gated sodium channel (Naᵥ) consist of large α subunits that associate with proteins, such as β subunits. An α subunit forms the core of the channel and is functional on its own. When the α subunit protein is expressed by a cell, it is able to form channels that conduct Na⁺ in a voltage-gated way, even if β subunits or other known modulating proteins are not expressed. When accessory proteins assemble with α subunits, the resulting complex can display altered voltage dependence and cellular localization.

Naᵥ have three main conformational states: closed, open and inactivated. Forward/back transitions between these states are correspondingly referred to as activation/deactivation (between open and closed, respectively), inactivation/reactivation (between inactivated and open, respectively), and recovery from inactivation/closed-state inactivation (between inactivated and closed, respectively). Closed and inactivated states are ion impermeable.

Before an action potential occurs, the axonal membrane is at its normal resting potential, about −70 mVin most human neurons, and Naᵥ are in their deactivated state, blocked on the extracellular side by their activation gates. In response to an increase of the membrane potential to about −55 mV (in this case, caused by an action potential), the activation gates open, allowing positively charged Na⁺ ions to flow into the neuron through the channels, and causing the voltage across the neuronal membrane to increase to +30 mV in human neurons. Because the voltage across the membrane is initially negative, as its voltage increases to and past zero (from −70 mV at rest to a maximum of +30 mV), it is said to depolarize. This increase in voltage constitutes the rising phase of an action potential.

At the peak of the action potential, when enough Na′ has entered the neuron and the membrane's potential has become high enough, the Naᵥ inactivate themselves by closing their inactivation gates. The inactivation gate can be thought of as a “plug” tethered to domains III and IV of the channel's intracellular alpha subunit. Closure of the inactivation gate causes Na′ flow through the channel to stop, which in turn causes the membrane potential to stop rising. The closing of the inactivation gate creates a refractory period within each individual Na′ channel. This refractory period eliminates the possibility of an action potential moving in the opposite direction back towards the soma. With its inactivation gate closed, the channel is said to be inactivated. With the Naᵥ no longer contributing to the membrane potential, the potential decreases back to its resting potential as the neuron repolarizes and subsequently hyperpolarizes itself, and this constitutes the falling phase of an action potential. The refractory period of each channel is therefore vital in propagating the action potential unidirectionally down an axon for proper communication between neurons. When the membrane's voltage becomes low enough, the inactivation gate reopens, and the activation gate closes in a process called ‘deinactivation’. With the activation gate closed and the inactivation gate open, the Na⁺ channel is once again in its deactivated state and is ready to participate in another action potential.

The proteins of these channels are named Naᵥ 1.1 through Naᵥ 1.9. The gene names are referred to as SCN1A through SCN11A (the SCN6/7A gene is part of the Nax sub-family and has uncertain function) (see Table 7). The individual sodium channels are distinguished not only by differences in their sequence but also by their kinetics and expression profiles.

TABLE 7
Human gene encoding Naᵥ protein isoforms

| Naᵥ protein | Human Gene encoding |
|---|---|
| isoforms | Naᵥ isoforms |
| Nav1.1 | SCN1A |
| Nav1.2 | SCN2A |
| Nav1.3 | SCN3A |
| Nav1.4 | SCN4A |
| Nav1.5 | SCN5A |
| Nav1.6 | SCN8A |
| Nav1.7 | SCN9A |
| Nav1.8 | SCN10A |
| Nav1.9 | SCN11A |
| NaX | SCN7A |

Naᵥ 1.4, which is encoded by the SCN4A gene is mainly expressed in skeletal muscle, and defect in the gene or its expression have been associated with human channelopathies such as hyperkalemic periodic paralysis, paramyotonia congenita, and potassium-aggravated myotonia.

Naᵥ 1.5, which is encoded by the SCN5A gene is mainly expressed in cardiac myocytes, uninnervated skeletal muscle, central neurons, gastrointestinal smooth muscle cells and interstitial cells of Cajal. Defects in the gene or its expression have been associated with human cardiac channelopathies such as Long QT syndrome Type 3, Brugada syndrome, progressive cardiac conduction disease, familial atrial fibrillation and idiopathic ventricular fibrillation; and gastrointestinal channelopathies such as irritable bowel syndrome.

“Antibodies” refers to immunoglobulin molecules and immunologically active portions of immunoglobulin molecules, which are molecules that contain an antigen binding site that immunospecifically binds an antigen. “Native antibodies” and “intact immunoglobulins”, or the like, are usually heterotetrameric glycoproteins of about 150,000 daltons, composed of two identical light (L) chains and two identical heavy (H) chains. The light chains from any vertebrate species can be assigned to one of two clearly distinct types, called kappa (κ) and lambda (λ), based on the amino acid sequences of their constant domains. Depending on the amino acid sequence of the constant domain of their heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA, and IgA2. The heavy-chain constant domains that correspond to the different classes of immunoglobulins are called α, δ, ε, γ, and μ, respectively. The subunit structures and three-dimensional configurations of different classes of immunoglobulins are well known. The intact antibody may have one or more “effector functions” which refer to those biological activities attributable to the Fc region (a native sequence Fc region or amino acid sequence variant Fc region or any other modified Fc region) of an antibody. Examples of antibody effector functions include C1q binding; complement dependent cytotoxicity; Fc receptor binding; antibody-dependent cell-mediated cytotoxicity (ADCC); phagocytosis; down regulation of cell surface receptors (e.g., B cell receptor (BCR); and cross-presentation of antigens by antigen presenting cells or dendritic cells.

Each light chain is linked to a heavy chain by one covalent disulfide bond, while the number of disulfide linkages varies among the heavy chains of different immunoglobulin isotypes. Each heavy and light chain also has regularly spaced intra-chain disulfide bridges. Each heavy chain has at one end a variable domain (VH) followed by a constant domain. Each light chain has a variable domain at one end (VL) and a constant domain at its other end; the constant domain of the light chain is aligned in space with the constant domain of the heavy chain, and the light-chain variable domain is aligned with the variable domain of the heavy chain. Particular amino acid residues are believed to form an interface between the light- and heavy-chain variable domains. Each variable region (of the heavy and light chain) includes three segments called complementarity-determining regions (CDRs) or hypervariable regions, and the more highly conserved portions of variable domains are called the framework region (FR). The variable domains of heavy and light chains each include four FR regions, largely adopting a β-sheet configuration, connected by three CDRs, which form loops connecting, and in some cases forming part of the β-sheet structure. The CDRs of the heavy and light chains are held together in close proximity by the FRs and, with the CDRs from the other chain, contribute to the formation of the antigen-binding site of antibodies (see Kabat et al., NIH Publ. No. 91-3242, Vol. I, pages 647-669 [1991]). The constant domains are not involved directly in binding an antibody to an antigen, but exhibit various effector functions, such as participation of the antibody in antibody dependent cellular cytotoxicity.

Experimentally, antibodies can be cleaved with the proteolytic enzyme papain, which causes each of the heavy chains to break, producing three separate antibody fragments. The two units that consist of a light chain and a fragment of the heavy chain approximately equal in mass to the light chain are called the Fab fragments (i.e., the “antigen binding” fragments). The third unit, consisting of two equal segments of the heavy chain, is called the Fc fragment. The Fc fragment is typically not involved in antigen-antibody binding but is important in later processes involved in ridding the body of the antigen.

Antibodies can be made, for example, via traditional hybridoma techniques, recombinant DNA methods, or phage display techniques using antibody libraries. For various other antibody production techniques, see Antibodies: A Laboratory Manual, eds. Harlow et al., Cold Spring Harbor Laboratory, 1988.

Antibody-derived scaffolds include VH domain, camelids (nanobodies or VHH), single chain variable fragments (scFv), antigen-binding fragments (Fab), avibodies, minibodies, CH2 domain (CH2D), abdurins, affibodies, adnectins, centryns, darpins and Fcabs. These scaffolds are attractive as platforms for developing novel therapeutics due to their smaller size (12-50 kDa) compared with IgG (150 kDa). The small size leads to greater and more rapid tissue accumulation and the ability to potentially recognize epitopes in protein targets not accessible to full size antibodies.

Single-domain antibody (sdAb), also known as a nanobody, is an antibody fragment consisting of a single monomeric variable antibody domain (VH). Nanobodies are small antigen-binding fragments (˜15 kDa) that are derived from heavy chain only antibodies present in camelids (VHH, from camels and llamas), and cartilaginous fishes (VNAR, from sharks). Nanobody V-like domains are useful alternatives to conventional antibodies due to their small size, and high solubility and stability across many applications. The first single-domain antibodies illustrated herein are engineered from heavy-chain antibodies found in camelids (VHH fragments). Single-domain camelid antibodies have been shown to be just as specific as a regular antibody and in some cases are more robust. VHH can easily be isolated using a phage panning procedure as used for traditional antibodies, allowing in vitro culture in large concentrations. The smaller size and single domain make these antibodies easier to transform into bacterial cells for bulk production.

A single-domain antibody is a polypeptide chain of about 100 to 200 amino acids, with one variable domain (VH) of a heavy-chain only antibody, or of a common IgG. These peptides have similar affinity to antigens as whole antibodies but are more heat-resistant and stable towards detergents and high concentrations of urea. Those derived from camelid and fish antibodies are less lipophilic and more soluble in water, possibly owing to their complementarity-determining region 3 (CDR3). The comparatively low molecular mass leads to a better permeability in tissues, and to a short plasma half-life since they are eliminated renally. Unlike whole antibodies, they do not show complement system triggered cytotoxicity because they lack an Fc region. Camelid and fish derived sdAbs are able to bind to hidden antigens that are not accessible to whole antibodies, for example to the active sites of enzymes. This property has been shown to result from their extended CDR3 loop, which is able to penetrate such buried sites

The major disadvantage of the smaller protein scaffolds is their rapid renal clearance and thus short circulating half-life. To address the short half-life disadvantage of the smaller antibody-like scaffolds, variants of the isolated human CH2 domain (IgG1 constant domain 2, CH2D or Abdurins) are developed as a new antibody-like scaffold platform. The human CH2D is small (˜12 kDa), is amenable to loop and β sheet modifications that can be used to generate large libraries of binders to target molecules, and yet retains a portion of the native FcRn binding site. Abdurins are isolated from the CH2 domain (heavy chain constant domain 2) and engineered to generate large libraries of binders to target molecules of interest. Importantly, Abdurins also retain a portion of the native FcRn binding motif which has been shown to bind FcRn protein, ensuring a circulating half-life in the 8-16 hour range.

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992) and Brennan et al., Science, 229:81 [1985]). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′2 fragments (Carter et al., Bio/Technology 10:163-167 [1992]). According to another approach, F(ab′) 2 fragments can be isolated directly from recombinant host cell culture. Other techniques for the production of antibody fragments will be apparent to the skilled practitioner. In other embodiments, the antibody of choice is a single chain Fv fragment (scFv). See WO 93/16185.

In various aspect, the invention provides a single-domain antibody (sdAb) that specifically and selectively binds to voltage-gated sodium channel (Nav)1.4 or Nav1.5.

In one aspect, the sdAb includes a complementarity-determining region (CDR) 1 having an amino acid sequence as set forth in SEQ ID NO:19, 22, 23, 26 or 29; a CDR2 having an amino acid sequence as set forth in SEQ ID NO:20, 24, 27 or 30; and a CDR3 having an amino acid sequence as set forth in SEQ ID NO:21, 25, 28 or 31.

In some aspects, the sdAb has a CDR1 having an amino acid sequence as set forth in SEQ ID NO:19, a CDR2 having an amino acid sequence as set forth in SEQ ID NO:20, and a CDR3 having an amino acid sequence as set forth in SEQ ID NO:21. For example, the sdAb can have the amino acid sequence of SEQ ID NO:5 or 8.

In other aspects, the sdAb has a CDR1 having an amino acid sequence as set forth in SEQ ID NO:23, a CDR2 having an amino acid sequence as set forth in SEQ ID NO:24, and a CDR3 having an amino acid sequence as set forth in SEQ ID NO:25. For example, the sdAb can have the amino acid sequence of SEQ ID NO:17.

Other exemplary sdAbs include sdAb having a CDR1 having an amino acid sequence as set forth in SEQ ID NO:22, a CDR2 having an amino acid sequence as set forth in SEQ ID NO:24, and a CDR3 having an amino acid sequence as set forth in SEQ ID NO:25. For example, the sdAb can have the amino acid sequence of SEQ ID NO:6 or 16.

Other exemplary sdAbs include sdAb having a CDR1 having an amino acid sequence as set forth in SEQ ID NO:26, a CDR2 having an amino acid sequence as set forth in SEQ ID NO:27, and a CDR3 having an amino acid sequence as set forth in SEQ ID NO:28. For example, the sdAb can have the amino acid sequence of SEQ ID NO:9 or 13.

Other exemplary sdAbs include sdAb having a CDR1 having an amino acid sequence as set forth in SEQ ID NO:29, a CDR2 having an amino acid sequence as set forth in SEQ ID NO:30, and a CDR3 having an amino acid sequence as set forth in SEQ ID NO:31. For example, the sdAb can have the amino acid sequence of SEQ ID NO:7, 10, 11, 12, 14, 15 or 18.

In one aspect, the amino acid sequence of the sdAb is set forth in SEQ ID NO:5 or 17.

sdAb can be derived from various sources. For example, sdAb can be camelid derived. Camelid antibodies are antibodies from the Camelidae family of mammals that include llamas, camels, and alpacas. These animals produce 2 main types of antibodies. One type of antibody camelids produce is the conventional antibody that is made up of 2 heavy chains and 2 light chains. They also produce another type of antibody that is made up of only 2 heavy chains. This is known as heavy chain IgG (hcIgG). While these antibodies do not contain the CH1 region, they retain an antigen binding domain called the VHH region. VHH antibodies, also known as single domain antibodies or Nanobodies®, contain only the VHH region from the camelid antibody. VHH antibodies can provide many benefits over traditional IgG antibodies. VHH antibodies are about 15 kDa in size compared to the 150 kDa size of an IgG antibody. Due to their smaller size they are able to detect certain epitopes that may not have been accessible with a traditional antibody due to steric hindrance. They are also able to penetrate tissue and enter cells easier allowing for more specific IHC staining and intracellular flow cytometry staining. The VHH antibodies are also more stable and can withstand a larger pH and temperature range.

Because they are derived from non-human sources, depending on their intended use, sdAb can be further modified. For example, the sdAb can be humanized.

An antibody or nanobody can be a “chimeric” antibody, in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855) [1984]). Chimeric antibody of interest can include “primatized” antibodies including variable domain antigen-binding sequences derived from a non-human primate (e.g., Old World Monkey, Ape etc.) and human constant region sequences; or “humanized” antibodies. Antibodies can also include regions from camel or llama in certain embodiments.

An antibody or nanobody can be a “humanized” form of non-human (e.g., camelid) antibodies, which are chimeric immunoglobulins, immunoglobulin chains or fragments thereof (such as Fv, Fab, Fab′, F(ab′)₂ or other antigen-binding subsequences of antibodies) containing minimal sequence derived from non-human immunoglobulin. For the most part, humanized antibodies are human immunoglobulins (recipient antibody) in which residues from a complementarity determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, humanized antibodies may comprise residues which are found neither in the recipient antibody nor in the imported CDR or framework sequences. These modifications are made to further refine and maximize antibody performance. In general, the humanized antibody will comprise substantially all of at least one, and typically two, variable domains, in which all or substantially all of the CDRs correspond to those of a non-human immunoglobulin and all or substantially all of the FRs are those of a human immunoglobulin sequence. The humanized antibody optimally also will comprise at least a portion of an immunoglobulin constant region (Fc), typically that of a human immunoglobulin. For further details, see Jones et al., Nature, 321:522-525 (1986); Reichmann et al., Nature, 332:323-329 (1988); and Presta, Curr. Op. Struct. Biol., 2:593-596 (1992). The humanized antibody includes a PRIMATIZED™ antibody wherein the antigen-binding region of the antibody is derived from an antibody produced by immunizing macaque monkeys with the antigen of interest.

In one aspect, the sdAb is selected from a camelid sdAb or a humanized sdAb. In some aspects, the sdAb is a llama sdAb or a humanized llama sdAb.

The sdAb of the invention has a high specificity and affinity for Naᵥ1.4 or Naᵥ1.5. by high “specificity”, it is meant that the sdAb recognizes its target with great precision, and that there is not cross-reactivity of the sdAb with other closely related antigen. For example, the sdAb of the invention specifically recognize Naᵥ1.4 or Naᵥ1.5, but do not recognize closely related antigen such as other Naᵥs. In one aspect, the sdAb does not bind to Naᵥ1.7 or Naᵥ1.9.

By high “affinity”, it is meant that the sdAb is capable or recognizing and binding to its antigen even in the presence of low concentration of the antigen. In other aspects, the sdAb binds to Naᵥ1.4 or Naᵥ1.5 with a nanomolar affinity.

In another embodiment, the invention provides an isolated polynucleotide encoding a sdAb that specifically binds to Naᵥ1.4 or Naᵥ1.5.

As used herein, the term “nucleic acid” or “oligonucleotide” refers to polynucleotides such as deoxyribonucleic acid (DNA) or ribonucleic acid (RNA). Nucleic acids include but are not limited to genomic DNA, cDNA, mRNA, iRNA, miRNA, tRNA, ncRNA, rRNA, and recombinantly produced and chemically synthesized molecules such as aptamers, plasmids, anti-sense DNA strands, shRNA, ribozymes, nucleic acids conjugated and oligonucleotides. According to the invention, a nucleic acid may be present as a single-stranded or double-stranded and linear or covalently circularly closed molecule. A nucleic acid can be isolated. The term “isolated nucleic acid” means, that the nucleic acid (i) was amplified in vitro, for example via polymerase chain reaction (PCR), (ii) was produced recombinantly by cloning, (iii) was purified, for example, by cleavage and separation by gel electrophoresis, (iv) was synthesized, for example, by chemical synthesis, or (vi) extracted from a sample. A nucleic might be employed for introduction into, i.e. transfection of, cells in the form of RNA which can be prepared by in vitro transcription from a DNA template. The RNA can moreover be modified before application by stabilizing sequences, capping, and polyadenylation.

Generally, nucleic acid can be extracted, isolated, amplified, or analyzed by a variety of techniques such as those described by Green and Sambrook, Molecular Cloning: A Laboratory Manual (Fourth Edition), Cold Spring Harbor Laboratory Press, Woodbury, NY 2,028 pages (2012); or as described in U.S. Pat. Nos. 7,957,913; 7,776,616; 5,234,809; U.S. Pub. 2010/0285578; and U.S. Pub. 2002/0190663.

In one aspect, the polynucleotide has an amino acid sequence as set forth in any of SEQ ID NOs:5-18. In other aspects, the polynucleotide has an amino acid sequence as set forth in SEQ ID NO:5 or 17.

While the polynucleotide can have a sequence as set forth in in any of SEQ ID NOs:5-18. Any polynucleotide sequence having certain sequence identity to the sequences provided herein are also included in the present disclosure. The terms “sequence identity” or “percent identity” are used interchangeably herein. To determine the percent identity of two polypeptide molecules or two polynucleotide sequences, the sequences are aligned for optimal comparison purposes (e.g., gaps can be introduced in the sequence of a first polypeptide or polynucleotide for optimal alignment with a second polypeptide or polynucleotide sequence). The amino acids or nucleotides at corresponding amino acid or nucleotide positions are then compared. When a position in the first sequence is occupied by the same amino acid or nucleotide as the corresponding position in the second sequence, then the molecules are identical at that position. The percent identity between the two sequences is a function of the number of identical positions shared by the sequences (i.e., % identity=number of identical positions/total number of positions (i.e., overlapping positions)×100). In some embodiments the length of a reference sequence (e.g., SEQ ID NO: 5-18) aligned for comparison purposes is at least 80% of the length of the comparison sequence, and in some embodiments is at least 90% or 100%. In an embodiment, the two sequences are the same length.

Ranges of desired degrees of sequence identity are approximately 80% to 100% and integer values in between. Percent identities between a disclosed sequence and a claimed sequence can be at least 80%, at least 85%, at least 90%, at least 95%, at least 96%, at least 97%, at least 98%, at least 99%, at least 99.5%, or at least 99.9%. In general, an exact match indicates 100% identity over the length of the reference sequence (e.g., SEQ ID NO: 5-18). Polypeptides and polynucleotides that are about 85, 86, 87, 88, 89, 90, 91, 92, 93, 94, 95, 96, 97, 98, 99 99.5% or more identical to polypeptides and polynucleotides described herein are embodied within the disclosure. For example, a polypeptide can have 80%, 85%, 90%, 91%, 92%, 93%, 94%, 95%, 96%, 97%, 98%, 99% or more identity to SEQ ID NO: 5-18.

Variants of the disclosed sequences also include peptides, or full-length protein, that contain substitutions, deletions, or insertions into the protein backbone, that would still leave at least about 70% homology to the original protein over the corresponding portion. A yet greater degree of departure from homology is allowed if like-amino acids, i.e. conservative amino acid substitutions, do not count as a change in the sequence. Examples of conservative substitutions involve amino acids that have the same or similar properties. Illustrative amino acid conservative substitutions include the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine, glutamine, or glutamate; methionine to leucine or isoleucine; phenylalanine to tyrosine, leucine or methionine; serine to threonine; threonine to serine; tryptophan to tyrosine; tyrosine to tryptophan or phenylalanine; valine to isoleucine to leucine.

In one embodiment, the invention provides an expression cassette including an isolated polynucleotide encoding a sdAb that specifically binds to Naᵥ1.4 or Naᵥ1.5.

The term “expression cassette” is used herein to refer to a recombinant nucleic acid construct that is manipulated by human intervention. A recombinant nucleic acid construct can contain two or more nucleotide sequences that are linked in a manner such that the product is not found in a cell in nature. In particular, the two or more nucleotide sequences can be operatively linked, such as one or more genes encoding one or more proteins of interest, one or more protein tags, functional domains and the like.

For example, the nucleic acid construct encodes at least one protein tag. A variety of protein tags are known in the art, such as epitope tags, affinity tags, solubility enhancing tags, and the like. Affinity tags are the most commonly used tag for aiding in protein purification while epitope tags aid in the identification of proteins. One of skill in the art would understand that some tags may be useful as more than one type of tag.

In one aspect, the expression cassette further includes a polynucleotide encoding a protein tag.

For example, the expression cassette can include a polynucleotide encoding a Histidine tag (6×-His) or a detectable tag, such as a fluorescent tag or a chemiluminescent tag.

In another aspect, the polynucleotide encoding the sdAb is operably linked to the polynucleotide encoding the protein tag to encode a fusion protein.

The terms “fusion protein” is meant to refer to a biologically active polypeptide, e.g., a sdAb, with or without a further effector molecule usually a protein or peptide sequence covalently linked (i.e., fused) by recombinant, chemical or other suitable method. If desired, the fusion molecule can be used at one or several sites through a peptide linker sequence. Alternatively, the peptide linker may be used to assist in construction of the fusion molecule. Specifically, illustrative fusion molecules are fusion proteins. Generally, fusion molecules also can include conjugate molecules.

In a specific embodiment the fusion protein of the present invention includes a sdAb with a cargo, for example, a binding protein or an enzyme or the catalytic domain of an enzyme. For example, the fusion could involve DNA that codes for a sdAb and DNA that codes for the catalytic domain of an E3 ligase. Other exemplary domains can include but are not limited to, for example, NEDL and HECT.

In a specific embodiment the fusion protein of the present invention includes a sdAb and a histidine tag.

In another embodiment, the invention provides a vector including an expression cassette including an isolated polynucleotide encoding a sdAb that specifically binds to Naᵥ1.4 or Naᵥ1.5.

The term “vector” or “expression vector” is used herein to refer to a recombinant nucleic acid construct including one or more nucleotide sequences operatively linked, such as one or more genes encoding one or more proteins of interest, one or more protein tags, functional domains, promoters and the like, for expression into hot cells. The expression vector of the invention can include regulatory elements controlling transcription generally derived from mammalian, microbial, viral or insect genes, such as an origin of replication to confer the vector the ability to replicate in a host, and a selection gene to facilitate recognition of transformants may additionally be incorporated. Those of skill in the art can select a suitable regulatory region to be included in such a vector depending on the host cell used to express the gene(s).For example, the expression vector usually comprises one or more promoters, operably linked to the nucleic acid of interest, capable of facilitating transcription of genes in operable linkage with the promoter. Several types of promoters are well known in the art and suitable for use with the present invention. The promoter can be constitutive or inducible.E3 ubiquitin-protein ligase NEDD4, also known as neural precursor cell expressed developmentally down-regulated protein 4 (whence “NEDD4”) is an enzyme that is, in humans, encoded by the NEDD4 gene. NEDD4 is an E3 ubiquitin ligase enzyme, that targets proteins for ubiquitination. NEDD4 is, in eukaryotes, a highly conserved gene, and the founding member of the NEDD4 family of E3 HECT ubiquitin ligases, which in humans consists of 9 members: NEDD4, NEDD4-2 (or NEDD4L), ITCH, SMURF1, SMURF2, WWP1, WWP2, NEDL1 (HECW1), NEDDL2 (HECW2). NEDD4 regulates a large number of membrane proteins, such as ion channels and membrane receptors, via ubiquitination and endocytosis.

For example, the fusion may include DNA that codes for a sdAb and a catalytic domain of an E3 ligase of the NEDD4 family. In one embodiment, the catalytic unit comprises the HECT domain of NEDD4L. In one embodiment, the catalytic unit comprises the HECT domain of WWP2.

Additional regulatory elements that may be useful in vectors, include, but are not limited to, polyadenylation sequences, translation control sequences (e.g., an internal ribosome entry segment, IRES), enhancers, or introns. Such elements may not be necessary, although they may increase expression by affecting transcription, stability of the mRNA, translational efficiency, or the like. Such elements can be included in a nucleic acid construct as desired to obtain optimal expression of the nucleic acids in the cell(s). Sufficient expression, however, may sometimes be obtained without such additional elements. Vectors also can include other elements. For example, a vector can include a nucleic acid that encodes a signal peptide such that the encoded polypeptide is directed to a particular cellular location (e.g., a signal secretion sequence to cause the protein to be secreted by the cell) or a nucleic acid that encodes a selectable marker. Non-limiting examples of selectable markers include puromycin, adenosine deaminase (ADA), aminoglycoside phosphotransferase (neo, G418, APH), dihydrofolate reductase (DHFR), hygromycin-B-phosphotransferase, thymidine kinase (TK), and xanthin-guanine phosphoribosyl transferase (XGPRT). Such markers are useful for selecting stable transformants in culture.

Non-limiting examples of vectors, suitable for use for the expression of high levels of recombinant proteins of interest include those selected from baculovirus, phage, plasmid, phagemid, cosmid, fosmid, bacterial artificial chromosome, viral DNA, Pl-based artificial chromosome, yeast plasmid, transposon, and yeast artificial chromosome. For example, the viral DNA vector can be selected from vaccinia, adenovirus, foul pox virus, pseudorabies and a derivative of SV40. Suitable bacterial vectors for use in practice of the invention methods include pQE70™, pQE60™, pQE-9™, pBLUESCRIPT™ SK, pHEN6, pBLUESCRIPT™ KS, pTRC99a™, pKK223-3™, pDR540™, PAC™ and pRIT2T™. Suitable eukaryotic vectors for use in practice of the invention methods include pWLNEO™, pXTI™, pSG5™, pSVK3™, pBPV™, pMSG™, and pSVLSV40™. Suitable eukaryotic vectors for use in practice of the invention methods include pWLNEO™, pXTI™, pSG5™, pSVK3™, pBPV™, pMSG™, and pSVLSV40™. One type of vector is a genomic integrated vector, or “integrated vector,” which can become integrated into the chromosomal DNA of the host cell. Another type of vector is an episomal vector, e.g., a nucleic acid capable of extra-chromosomal replication. Viral vectors include adenovirus, adeno-associated virus (AAV), retroviruses, lentiviruses, vaccinia virus, measles viruses, herpes viruses, and bovine papilloma virus vectors (see, Kay et al., Proc. Natl. Acad. Sci. USA 94:12744-12746 (1997) for a review of viral and non-viral vectors). Viral vectors are modified so the native tropism and pathogenicity of the virus has been altered or removed. The genome of a virus also can be modified to increase its infectivity and to accommodate packaging of the nucleic acid encoding the polypeptide of interest.

In an additional embodiment, the invention provides a host cell including a polynucleotide encoding a sdAb that specifically binds to Naᵥ1.4 or Naᵥ1.5, expression cassette including a polynucleotide encoding a sdAb that specifically binds to Naᵥ1.4 or Naᵥ1.5, or a vector including an expression cassette including an isolated polynucleotide encoding a sdAb that specifically binds to Naᵥ1.4 or Naᵥ1.5.

The nucleic acid construct (or the vector) of the present invention may be introduced into a host cell to be altered thus allowing expression of the protein within the cell. A variety of host cells are known in the art and suitable for proteins expression and extracellular vesicles production. Examples of typical cell used for transfection include, but are not limited to, a bacterial cell, a eukaryotic cell, a yeast cell, an insect cell, or a plant cell. For example, E. coli, Bacillus, Streptomyces, Pichia pastoris, Salmonella typhimurium, Drosophila S2, Spodoptera SJ9, CHO, COS (e.g. COS-7), 3T3-F442A, HeLa, HUVEC, HUAEC, NIH 3T3, Jurkat, 293, 293H, or 293F.

The nucleic acid construct of the present invention, included into a vector, may be introduced into a cell to be altered thus allowing expression of the chimeric protein within the cell. A variety of methods are known in the art and suitable for introduction of nucleic acid into a cell, including viral and non-viral mediated techniques. Examples of typical non-viral mediated techniques include, but are not limited to, electroporation, calcium phosphate mediated transfer, nucleofection, sonoporation, heat shock, magnetofection, liposome mediated transfer, microinjection, microprojectile mediated transfer (nanoparticles), cationic polymer mediated transfer (DEAE-dextran, polyethylenimine, polyethylene glycol (PEG) and the like) or cell fusion. Other methods of transfection include proprietary transfection reagents such as LIPOFECTAMINE™, DOJINDO HILYMAX™, FUGENE™, JETPEI™, EFFECTENE™ and DREAMFECT™.

In one embodiment, the invention provides a pharmaceutical composition including the sdAb described herein and a pharmaceutically acceptable carrier.

As used herein, “pharmaceutical composition” refers to a formulation comprising an active ingredient, and optionally a pharmaceutically acceptable carrier, diluent or excipient. The term “active ingredient” can interchangeably refer to an “effective ingredient” and is meant to refer to any agent that is capable of inducing a sought-after effect upon administration. In one embodiment, the active ingredient includes a biologically active molecule. The biologically active molecule of the present invention is a sdAb that specifically binds to Naᵥ1.4 or Naᵥ1.5. By “pharmaceutically acceptable” it is meant the carrier, diluent or excipient must be compatible with the other ingredients of the formulation and not deleterious to the recipient thereof, nor to the activity of the active ingredient of the formulation. Pharmaceutically acceptable carriers, excipients or stabilizers are well known in the art, for example Remington's Pharmaceutical Sciences, 16th edition, Osol, A. Ed. (1980). Pharmaceutically acceptable carriers, excipients, or stabilizers are nontoxic to recipients at the dosages and concentrations employed, and may include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (for example, Zn-protein complexes); and/or non-ionic surfactants such as TWEEN™, PLURONICS™ or polyethylene glycol (PEG). Examples of carrier include, but are not limited to, liposome, nanoparticles, ointment, micelles, microsphere, microparticle, cream, emulsion, and gel. Examples of excipient include, but are not limited to, anti-adherents such as magnesium stearate, binders such as saccharides and their derivatives (sucrose, lactose, starches, cellulose, sugar alcohols and the like) protein like gelatin and synthetic polymers, lubricants such as talc and silica, and preservatives such as antioxidants, vitamin A, vitamin E, vitamin C, retinyl palmitate, selenium, cysteine, methionine, citric acid, sodium sulfate and parabens. Examples of diluent include, but are not limited to, water, alcohol, saline solution, glycol, mineral oil and dimethyl sulfoxide (DMSO).

Voltage-gated sodium channels (Naᵥ) are responsible for the fast rise of action potentials in excitable tissues. Dysfunction of Naᵥ proteins caused by mutations have been implicated in several human genetic diseases such as hypokalemic periodic paralysis, myotonia and Brugada syndrome. Despite the physiological importance of Naᵥ channels, development of antibodies specific for the different Naᵥ isoforms has been challenging, rendering the discovery of isoform-specific antibodies that recognize Naᵥ channels with nM or higher affinity and without cross-reactivity highly desirable. The sdAb described herein are the foundation for developing reagents such as bait proteins to capture and purify Naᵥs from cell lysates, as crystallization chaperones, as molecular Naᵥ visualization agents and as Naᵥ channel modulators for tissue-specific targeting in health and disease. The sdAb described herein can be used as molecular Naᵥ visualization agents (for western blot, ELISA, live cell imaging), bait proteins to capture and purify Naᵥs from cell lysates, Naᵥ channel modulators for tissue-specific targeting in health and disease, and crystallization chaperones. sdAbs that recognize molecular targets (such as Naᵥ1.4 and Naᵥ1.5) implicated in diseases are molecular probes that are a sought-after reagent for the study and diagnose of myotonias and cardiac arrhythmia for example. They have demonstrated usefulness for highly specific detection and affinity capture of endogenous and recombinant Naᵥ1.4 (primary skeletal muscle isoform) and Naᵥ1.5 (primary cardiac isoform) proteins without cross-reactivity to Naᵥ1.7 and Naᵥ1.9 (predominant in neurons). Isoform-specific study of Naᵥ channels using nanobodies has not been achieved previously.

In another embodiment, the invention provides a method of detecting and/or capturing Naᵥ1.4 or Naᵥ1.5 in a sample including contacting the sample with the sdAb described herein; and detecting and/or capturing a complex between the sdAb and the Naᵥ1.4 or Naᵥ1.5.

By “detecting” it is meant that the methods allow for the identification of the Naᵥ1.4 or Naᵥ1.5 in a sample (i.e., the method allow to assert if Naᵥ1.4 or Naᵥ1.5 is present or absent in the sample). In one aspect, detecting the complex is by western blot, immunohistochemistry, flow cytometry, enzyme-linked immunosorbent assay (ELISA) or immunofluorescence.

By “capturing”, it is meant that the sdAb allows for the separation of Naᵥ1.4 or Naᵥ1.5 from the sample. The channels can for example be purified or isolated from a sample using the sdAb described herein. For example, using a sdAb fused to a protein tag that can be easily separated from a sample. In other aspects, capturing the complex is by immunoprecipitation (IP) or co-IP.

As used herein, a “sample” or “biological sample” is meant to refer to any “biological specimen” collected from a subject, and that is representative of the content or composition of the source of the sample, considered in its entirety. A sample can be collected and processed directly for analysis or be stored under proper storage conditions to maintain sample quality until analyses are completed. Ideally, a stored sample remains equivalent to a freshly collected specimen. The source of the sample can be an internal organ, vein, artery, or even a fluid. Non-limiting examples of sample include blood, plasma, urine, saliva, sweat, organ biopsy, a tissue biopsy, a cell, cerebrospinal fluid (CSF), tear, semen, vaginal fluid, feces, skin, breast milk, and hair. Specifically, the present invention relies on the use of any biological sample collected from a subject that is susceptible to contain and/or express Naᵥ1.4 or Naᵥ1.5.

In one aspect, the sample is a tissue or cell derived from a cardiac tissue, a skeletal muscle tissue, a nerve tissue or a lysate thereof.

In other aspects, the sample is from a tissue or cell from a subject who has cancer.

The methods described herein can be performed on cells or tissue directly, when the integrity of the cell or tissue is to be maintained (for example to assess tissue, cellular or subcellular localization of Naᵥ1.4 or Naᵥ1.5). The methods can also be performed on lysates of the cell or tissue, when no tissue or cell localization is to be assessed. A lysate refers to a preparation of the sample (tissue or cell) to result in a homogeneous solution. For example, the cell or tissue can be lysed, to provide a homogeneous cell solution or tissue solution.

The cell can be an adherent fixed and permeabilized cell, a suspension of fixed and permeabilized cells, or a cell lysate coated on a surface. The tissue can be fixed and preserved such as formalin fixed paraffin embedded, and tissue sections can be prepared on cover glass or equivalent material.

For example, the cell can be cultured on a cover glass or equivalent material, and fixed and permeabilized when the appropriate confluence is reached. Using fluorescently labeled sdAb in a classic immunofluorescent assay, the immune complexes between the sdAb and Naᵥ1.4 or Naᵥ1.5 can be detected by fluorescence by immunofluorescent microscopy.

The cells can be cultured until the required number of cells is reached, the cells can then be collected in a suspension, fixed and permeabilized. Using fluorescently labeled sdAb in a classic immune assay, the immune complexes can be detected by observing the fluorescence by flow cytometry.

The cells can be cultured until the required number of cells is reached, the cells can then be collected in a suspension, fixed and lysed, or the tissue can be collected and lysed to obtain a cell lysate or a tissue lysate. Using tagged sdAb in an immunoprecipitation or co-immunoprecipitation assays immune complexes between the sdAb and Naᵥ1.4 or Naᵥ1.5 can be captured from the cell lysate. Alternatively, using chemiluminescently labeled sdAb in a western blot assay, the immune complexes between the sdAb and Naᵥ1.4 or Naᵥ1.5 can be detected and the presence and quantity of Naᵥ1.4 or Naᵥ1.5 in the cell lysate or tissue lysate can be estimated.

In one embodiment, the invention provides a method of detecting a disease or condition in a subject including contacting a sample from the subject with the composition described herein and detecting the sdAb in the sample.

By “detecting a disease or condition” it is meant that the sdAb of the invention can be used to diagnose a disease in a subject based on the analysis of a sample collected form the subject. Based on the specificity and sensitivity of the sdAb of the present invention, by contacting the sdAb with a sample collected from a subject, the presence (or absence) and the localization of Naᵥ1.4 or Naᵥ1.5 in the sample can indicate that the subject has a disease or condition related to the expression of Naᵥ1.4 or Naᵥ1.5.

For example, an absence of detection of Naᵥ1.4 in a skeletal muscle sample obtained from a subject (where Naᵥ1.4 is mainly expressed) can indicate a defect in the expression of SCN4A in the subject, and therefore indicate that the subject has or is at risk of having a channelopathy such as hyperkalemic periodic paralysis, paramyotonia congenita, or potassium-aggravated myotonia.

Similarly, an absence of detection of Naᵥ 1.5 in cardiac myocytes, uninnervated skeletal muscle, central neurons, gastrointestinal smooth muscle cells or interstitial cells of Cajal (where Naᵥ 1.5 is mainly expressed) can indicate a defect in the expression of SCN5A in the subject, and therefore indicate that the subject has or is at risk of having a cardiac channelopathy such as Long QT syndrome Type 3, Brugada syndrome, progressive cardiac conduction disease, familial atrial fibrillation and idiopathic ventricular fibrillation; or a gastrointestinal channelopathy such as irritable bowel syndrome.

The term “subject” as used herein refers to any individual or patient to which the subject methods are performed. Generally, the subject is human, although as will be appreciated by those in the art, the subject may be an animal. Thus other animals, including vertebrate such as rodents (including mice, rats, hamsters and guinea pigs), cats, dogs, rabbits, farm animals including cows, horses, goats, sheep, pigs, chickens, etc., non-human primate and primates (including monkeys, chimpanzees, orangutans and gorillas) are included within the definition of subject.

In one aspect, the disease or condition is selected from the group consisting of cardiac arrhythmia, myotonia, neuropathic pain, hypokalemic periodic paralysis, Long QT syndrome, sudden cardiac death syndrome and Brugada syndrome.

In other aspects, the disease or condition is selected from the group consisting of colon, prostate, breast, cervical, lung, pancreas, biliary, rectal, liver, kidney, testicular, brain, head and neck, ovarian cancer, melanoma, sarcoma, multiple myeloma, leukemia, and lymphoma.

The term “cancer” refers to a group of diseases characterized by abnormal and uncontrolled cell proliferation starting at one site (primary site) with the potential to invade and to spread to other sites (secondary sites, metastases) which differentiate cancer (malignant tumor) from benign tumor. Virtually all the organs can be affected, leading to more than 100 types of cancer that can affect humans. Cancers can result from many causes including genetic predisposition, viral infection, exposure to ionizing radiation, exposure environmental pollutant, tobacco and or alcohol use, obesity, poor diet, lack of physical activity or any combination thereof. As used herein, “neoplasm” or “tumor” including grammatical variations thereof, means new and abnormal growth of tissue, which may be benign or cancerous. In a related aspect, the neoplasm is indicative of a neoplastic disease or disorder, including but not limited, to various cancers.

Exemplary cancers described by the national cancer institute include: Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood: Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia; Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma. Childhood Brain Stem; Glioma. Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's; Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplasia Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood', Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland' Cancer, Childhood; Sarcoma, Ewing's Family of Tumors; Sarcoma, Kaposi's; Sarcoma (OsteosarcomaVMalignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumor.

In another embodiment, the invention provides a method of treating cardiac arrhythmia, myotonia or sudden cardiac death syndrome in a subject including administering to the subject a single-domain antibody (sdAb) that specifically binds to voltage-gated sodium channel (Naᵥ)1.4 or Naᵥ1.5 for tissue-specific targeting of Naᵥ1.4 or Naᵥ1.5.

As used herein the sdAb that specifically binds to Naᵥ1.4 or Naᵥ1.5 can interact with and modulate the activity of a Naᵥ1.4 or Naᵥ1.5 channel. For example, a sdAb that specifically binds to Naᵥ1.4 or Naᵥ1.5 can prevent a Naᵥ from transitioning between one state and the other (i.e., open, closed, and inactivated). A sdAb that specifically binds to Naᵥ1.4 or Naᵥ1.5 can modulate or inhibit the transition between activation and deactivation, inactivation and reactivation or between recovery from inactivation and closed-state inactivation.

The term “treatment” is used interchangeably herein with the term “therapeutic method” and refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The terms “administration of” and or “administering” should be understood to mean providing a pharmaceutical composition in a therapeutically effective amount to the subject in need of treatment. Administration routes can be enteral, topical or parenteral. As such, administration routes include but are not limited to intracutaneous, subcutaneous, intravenous, intraperitoneal, intraarterial, intrathecal, intracapsular, intraorbital, intracardiac, intradermal, transdermal, transtracheal, subcuticular, intraarticulare, subcapsular, subarachnoid, intraspinal and intrasternal, oral, sublingual buccal, rectal, vaginal, nasal ocular administrations, as well infusion, inhalation, and nebulization. The phrases “parenteral administration” and “administered parenterally” as used herein means modes of administration other than enteral and topical administration. The pharmaceutical compositions can be administered in a variety of unit dosage forms depending upon the method of administration. Suitable unit dosage forms, include, but are not limited to powders, tablets, pills, capsules, lozenges, suppositories, patches, nasal sprays, injectables, implantable sustained-release formulations, lipid complexes, etc.

The pharmaceutical composition may also contain other therapeutic agents, and may be formulated, for example, by employing conventional vehicles or diluents, as well as pharmaceutical additives of a type appropriate to the mode of desired administration (for example, excipients, preservatives, etc.) according to techniques known in the art of pharmaceutical formulation.

In one aspect, the sdAb is a sdAb as described herein, that specifically binds to Naᵥ1.4 or Naᵥ1.5. For example, the sdAb can be a sdAb having an amino acid sequence as set forth in one of SEQ ID NO:5-18. In one aspect, the sdAb has an amino acid sequence as set forth in SEQ ID NO:5 or 17.

In an additional embodiment, the invention provides a method of treating cancer in a subject including administering to the subject a sdAb that specifically binds to Naᵥ1.4 or Naᵥ1.5 for tissue-specific targeting of Naᵥ1.4 or Naᵥ1.5.

In one aspect, the cancer is selected from the group consisting of colon, prostate, breast, cervical, lung, pancreas, biliary, rectal, liver, kidney, testicular, brain, head and neck, ovarian cancer, melanoma, sarcoma, multiple myeloma, leukemia, and lymphoma.

In another aspect, the sdAb is a sdAb as described herein, that specifically binds to Naᵥ1.4 or Naᵥ1.5. For example, the sdAb can be a sdAb having an amino acid sequence as set forth in one of SEQ ID NO:5-18. In aspect, the sdAb has an amino acid sequence as set forth in SEQ ID NO:5 or 17.

Presented below are examples discussing sdAb that specifically binds Naᵥ1.4 or Naᵥ1.5 contemplated for the discussed applications. The following examples are provided to further illustrate the embodiments of the present invention but are not intended to limit the scope of the invention. While they are typical of those that might be used, other procedures, methodologies, or techniques known to those skilled in the art may alternatively be used

### EXAMPLES

### Example 1

### Material and Methods

C-Terminal Naᵥ1.4-CaM Protein Expression for Llama Immunization

The GST-tagged C-terminal region of Naᵥ1.4 (aa 1599-1764), in complex with CaM (CTNaᵥ1.4T-CaM) was expressed and purified from BL21-CodonPlus RIL (Agilent) E. coli cells using a GST-sepharose 4b resin (GE Lifesciences) followed by anion exchange chromatography and a final gel filtration chromatography as described by Yoder et al. Purified CTNaᵥ1.4T-CaM at 56 mg/ml was used for generation of single chain antibodies by llama immunization.

Llama Immunization and Construction of the Library (Naᵥ1.4CaM)

The immunization protocol and the construction of the library were done as previously described. In brief, two llamas (Lama glama) were immunized three times intramuscularly every 15 days with 100 μg of purified CTNaᵥ1.4T-CaM emulsified with complete Freund's adjuvant (Sigma). The humoral immune response in the sera was monitored by ELISA performed on MaxiSorp plates (Thermo scientific) coated with CTNaᵥ1.4T-CaM. 45 Days after the first immunization (D45), the animals were bled (Table 1). Peripheral blood monocyte cells (PBMCs) were isolated from 300 ml of blood by Ficoll-Paque (GE Healthcare) gradient centrifugation.

TABLE 1
Quantification of total mRNA isolated from PBMB
cells following immunization of Llama #1
and #2 at 46 or 48 days (D 46 or D 48)

| | | Absorbance | Absorbance |
|---|---|---|---|
| Sample | Concentration (ng/μl) | ratio 260/280 | ratio 260/230 |
| #1 D 46 C1 | 295.5 | 1.99 | 1.36 |
| #1 D 46 C2 | 104.4 | 2.10 | 2.27 |
| #1 D 48 | 400.2 | 2.01 | 2.26 |
| #2 D 46 C1 | 286.9 | 2.11 | 2.20 |
| #2 D 46 C2 | 766.5 | 2.06 | 2.27 |
| #2 D 48 | 694.7 | 2.01 | 2.29 |

Total RNA was purified from these cells using the RNeasy midi Kit (Qiagen) and subjected to cDNA synthesis. The Nb coding regions were amplified by PCR using specific primers: forward (fwd_001)=5′ GTCCTGGCTGCTCTTCTACAAGG 3′ (SEQ ID NO:1); reverse (rvd_002): 5′ GGTACGTGCTGTTGAACTGTTCC 3′ (SEQ ID NO:2). The amplicons were purified from agarose gels, digested with PstI and NotI (Roche) and cloned into the pHEN4 phagemid vector downstream of the PelB-leader peptide and upstream of the HA tag. E. coli TG1 (Lucigen) cells were transformed with this vector obtaining two different libraries (one for each llama). Fifteen clones randomly chosen from each library were used for plasmid DNA preparation (QIAprep Spin Miniprep Kit, QIAGEN) and run on agarose gel electrophoresis for qualitative analysis where >70% of clones were found to be positive for Nbs.

Phage Display Selection of Naᵥ1.4-CaM-Specific Nbs and Subcloning

The panning was performed in MaxiSorp plates. Briefly, wells were sensitized with either 1 μg of CTNaᵥ1.4T-CaM+1 mM DTT or uncoated to serve as negative controls. After blocking with 3% skim milk in PBS, 1×10¹² phages in PBS were added to each test and control coated wells and incubated for 2 h at RT. Wells were then extensively washed with 25 mM Tris, 150 mM NaCl, 1 mM DTT, 0.05% Tween 20, pH 8.0, and bound phages were eluted with 0.25 mg/ml trypsin (Gibco). Eluted phages were titrated and subjected to rounds of panning following the same procedure. Output phage titers were estimated by infection of TG1 cells and plating them on LB with 100 μg/ml Amp and 2% glucose. A total of 87 randomly chosen clones were grown in deep well plates (Greiner bio-one) containing 1 ml of 2×TY medium added with 100 μg/ml Amp and 0.1% glucose for 3 h at 37° C. and 200 rpm until cell growth reached the exponential phase. The expression of Nbs was induced by adding 1 mM IPTG per well with shaking at 200 rpm for 4 h at 37° C. The Nbs were obtained from the periplasm and were tested by ELISA on MaxiSorp plates sensitized with 1 μg of CTNaᵥ1.4T-CaM and 1 mM DTT. After washing with 25 mM Tris, 150 mM NaCl, 1 mM DTT, 0.05% Tween 20, pH 8.0, Nbs were detected by incubation with a secondary antibody (anti-HA high affinity, Roche) and developed using anti-rat IgG peroxidase-conjugate (Sigma). Fourteen positive clones were selected for sequencing using universal M13 reverse as primer, and then classified in families based on the length and variability of their CDR3. Multiple sequence alignments were done with Clustal Omega. One representative clone for each family was selected for periplasmic Nb expression, purification and further characterization.

To determine whether these fourteen Nbs recognize CTNaᵥ1.4 or CaM, periplasmic-extract ELISA (PE-ELISA) was performed. MaxiSorp plates were sensitized with 0.1 μg of either CTNaᵥ1.4T-CaM, Ca2+CaM or apoCaM for 2 h at RT and blocked with 5% Bovine Serum Albumin (BSA, Sigma) for 2 h at RT. 50 μl of a 1:100 dilution of E. coli TG1 periplasm extracts expressing each Nb (Nb17, Nb26, Nb30 and Nb82) were incubated overnight at 4° C.). Nbs were detected by incubation with an anti-HA high affinity secondary antibody (Roche) and the ELISA was developed using an anti-rat IgG peroxidase-conjugated antibody (Sigma). Nb17, Nb30 and Nb82 were then subcloned in pHEN6. The coding regions were amplified by PCR using the following specific primers: forward (A6E) 5′ GAT GTG CAG CTG CAG GAG TCT GGR GGA GG 3′ (SEQ ID NO:3); reverse (p38): 5′ GGA CTA GTG CGG CCG CTG GAG ACG GTG ACC TGG GT 3′ (SEQ ID NO:4). The amplicons were purified from agarose gels, digested with restriction enzymes PsTI and BstEII (New England Biolabs) and cloned into the pHEN6-TEV-His vector for periplasmic expression of the recombinant Nb protein in E. coli Rosetta-gami 2 (DE3) cells. Nb17 and Nb82 were also subcloned into the vector pcDNA3.1 with a C-terminal GFP-tag (Genscript, NY).

Purification of Nb17 and Nb82

The expression and purification of the Nbs was performed as described previously with minor modifications. Transformed E. coli Rosetta-gami 2 (DE3) cells were grown in LB medium supplemented with antibiotics in addition to 1 mM MgCl2 and 0.1% glucose.

Cultures were induced with 1 mM IPTG at OD₆₀₀=0.8-0.9 and incubated overnight at 18° C. at 200 rpm. Cells were harvested by centrifugation and frozen at −80° C. for at least 2 h before proceeding to thawing and osmotic shock to extract the periplasmic proteins. For this, cells were thawed in a water bath, re-suspended in osmotic-shock-TES-buffer (0.2 M Tris-HCl, 0.65 mM EDTA, 0.5 M sucrose, pH 8.0) and incubated at 4° C. for 1 h on an orbital shaker. Then, cells were further diluted with osmotic-shock-TES-buffer and incubated at 4° C. for 45 min on an orbital shaker. The periplasmic fraction was isolated by centrifugation at 8000 rpm at 4° C. for 30 min using an SLA1500 rotor. To the filtered supernatant (0.22 μm PES filter), 2.5 mM TCEP and 5 mM imidazole were added and incubated ON with 1 ml of pre-washed Ni-NTA agarose Superflow beads at 4° C. using an orbital shaker. The Ni-NTA agarose beads were equilibrated with 50 mM Tris, 150 mM NaCl, pH 8.0 (TN buffer). Beads were washed in a gravity flow column using 40 ml of TN buffer pH 8.0 with 10 mM imidazole and Nbs were eluted using 40 ml of TN buffer pH 8.0 with 300 mM imidazole. Purification fractions were analyzed by SDS-PAGE and the Nb-containing elution fractions were pooled, dialyzed at 4° C. into low salt TN buffer (20 mM Tris-HCl, 50 mM NaCl, 2 mM DTT, pH 7.5) and concentrated to 1 mg/ml before loading on a Superdex 75 (10/300) gel filtration column using TN buffer. The peak fractions from gel filtration containing the Nbs were then concentrated using a 3.5 kDa cut-off filtering device to a final concentration of ˜10-11 mg/ml as 24 μl aliquots and flash frozen and stored at −80° C.

Purification of C-Terminal Regions of Naᵥ1.4, Naᵥ1.5, Naᵥ1.7, and Naᵥ1.9 in Complex with CaM (CTNaᵥ1.X-CaM) for ELISA and Binding Experiments

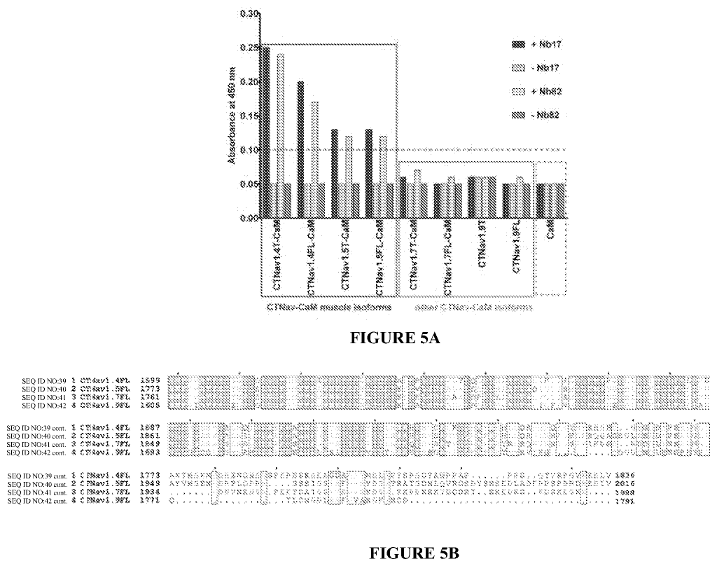

The constructs of C-terminal regions of the voltage gated sodium channel isoforms were named consistently as ‘truncated, (T)’ for constructs ending at equivalent residue of Naᵥ1.41764, and ‘full length, (FL)’ for constructs ending at equivalent residue of Naᵥ1.41836. The constructs are CTNaᵥ1.4T with amino acids 1599-1764, CTNaᵥ1.4FL aa 1599-1836, CTNaᵥ1.5T aa 1773-1940, CTNaᵥ1.5FL aa 1773-2016, CTNaᵥ1.7T aa 1761-1928 CTNaᵥ1.7FL aa 1761-1988, CTNaᵥ1.9T aa 1605-1768, CTNaᵥ1.9FL aa 1605-1791 (Table 2, FIG. 5B).

TABLE 2
Amino acid included in the CTNaᵥ constructs

| CTNaᵥ constructs | Residues includes in the constructs |
|---|---|
| CTNaᵥ 1.4T | 1599-1764 |
| CTNaᵥ 1.4FL | 1599-1836 |
| CTNaᵥ 1.5T | 1773-1940 |
| CTNaᵥ 1.5FL | 1773-2016 |
| CTNaᵥ 1.7T | 1761-1928 |
| CTNaᵥ 1.7FL | 1761-1988 |
| CTNaᵥ 1.9T | 1605-1768 |
| CTNaᵥ 1.9FL | 1605-1791 |

Each construct was co-expressed with mammalian Calmodulin (CaM) and purified from BL21-CodonPlus RIL (Agilent) E. coli cells using a GST-sepharose column followed by anion exchange chromatography and a final gel filtration chromatography step as described by Yoder et al. with minor modifications. In brief, cells were grown overnight at 37° C. in 100 ml of LB medium supplemented with 50 μg/ml kanamycin, 20 μg/ml chloramphenicol and 100 μg/ml carbenicillin. Ten ml of the overnight culture was used to inoculate 1 1 of LB media containing the same antibiotics. The cells were grown at 37° C. to an OD_{600 nm}=0.8-0.9 and protein expression was induced with 1 mM IPTG. The cells were grown overnight at 18° C. (approximately 18 h), centrifuged and the cell pellet was frozen at −80° C. After thawing, pellets were re-suspended with PBS at 5× volume/weight ml/g of cells. DNAse was added and the cells were lysed using a microfluidizer (Microfluidics Corporation; model 110 Y) and the lysate clarified at 27,500×g. The supernatant was loaded on to a 3 ml Glutathione Sepharose 4 Fast Flow resin (GST resin) using gravity flow. The column was washed with 30 ml wash buffer (PBS added with 100 mM NaCl) and free CaM was purified from this fraction for other experiments. The CTNaᵥT-CaM and CTNaᵥFL-CaM complexes were eluted in aliquots of 5 ml with an elution buffer containing 10 mM reduced L-glutathione in 50 mM

Tris-HCl, pH 8.0. Eluted fractions containing protein were pooled and 5 μg of PreScission protease was added per mg of CTNaᵥ-CaM for cleaving the GST-tag. Dialysis was performed against 21 of buffer containing 20 mM Tris, 50 mM NaCl, 1 mM DTT, pH 7.4. The buffer was changed twice, and the final dialysis was allowed to proceed overnight at 4° C. The dialyzed and PreScission protease-cleaved protein was loaded on a 15 ml Source Q anion exchange column (GE). Elution was performed using buffer 20 mM Tris, 1 mM DTT, pH 7.4 and a gradient of 50-500 mM NaCl. Free, cleaved GST eluted at −8 mS/cm and CTNaᵥ-CaM complexes eluted at between 14-27 mS/cm conductance that varied depending on the Naᵥ isoform. Fractions were judged to be >95% pure by SDS-PAGE gel then pooled and concentrated to −15-20 mg/ml protein and flash frozen and stored at −80° C. In the case of CTNaᵥ1.9T and CTNaᵥ1.9FL, CaM did not co-elute with the CTNaᵥs unlike the other cases.

Detection of Nb Specificity for Naᵥs by ELISA

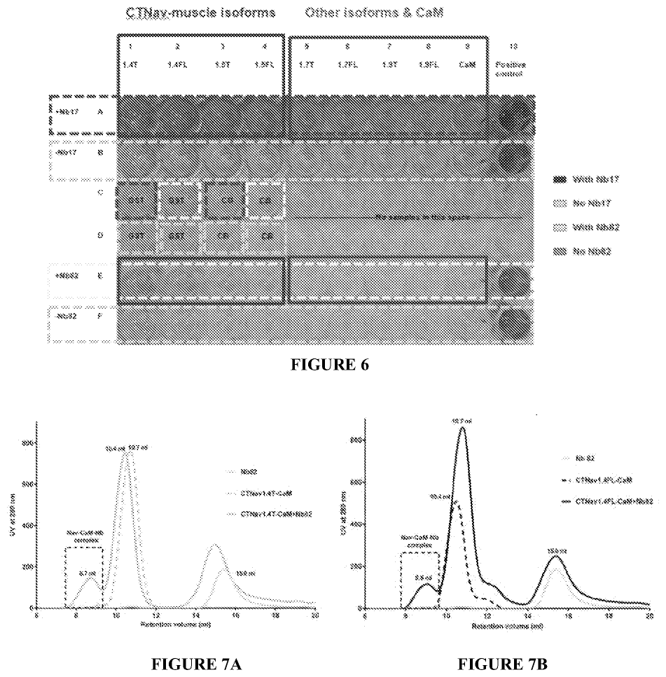

Purified Nb-His-tagged proteins were assessed for recognition of Naᵥ proteins in high-binding 96-well EIA/RIA plates (Costar-9018). The analytes, purified CTNaᵥT-CaM and CTNaᵥFL-CaM protein isoforms (CTNaᵥ1.4T-CaM, CTNaᵥ1.4FL-CaM, CTNaᵥ1.5T-CaM, CTNaᵥ1.5FL-CaM, CTNaᵥ1.7T-CaM, CTNaᵥ1.7FL-CaM), CTNaᵥ1.9T, CTNaᵥ1.9FL, CaM, GST and His-tagged positive control protein (scFv) were diluted in PBS and coated (1 μg/well) to a 96-well plate as shown in the template (FIG. 6) using carbonate-bicarbonate buffer, pH 9.5, at 4° C. overnight. Next, the plate was washed 5 times using 100 μl/well wash buffer (PBS added with 0.1% Tween-20). Next, protein coated wells were incubated with 100 μl/well of 0.01 μg of Nb17 or Nb82 diluted in 1× blocking buffer (PBS added with 1% non-fat milk) for 2 h at RT on a shaking platform. The plates were then washed 5 times using 100 μl/well of wash buffer and incubated with mouse anti-His6-peroxidase secondary antibody (Roche) at 100 mU/ml in blocking buffer for 1 h at RT. The plates were finally washed in wash buffer 5 times and peroxidase activity was assayed using 100 μl/well o-phenyl diamine in 150 mM citrate phosphate buffer and 30% H₂O₂. The plates were incubated in the dark for a few minutes until a yellow color developed in at least one of the wells. The reaction was stopped by the addition of 100 μl/well of 2 N H₂SO₄ and the absorbance was read at 450 nm (20 flashes) using a Tecan infinite M1000 micro plate reader (Tecan i-control, 2.0.10.0 application).

Crystallization of Nb82

Purified Nb82 was used at 10 mg/ml for all crystallization experiments. Sparse matrix commercial crystallization screens were used to find conditions in hanging-drop, vapor diffusion by mixing equal volumes of Nb82 and reservoir solution. Nb82 crystallized in 2% (w/v) PEG MME 550, 1.8 M ammonium sulfate, 0.1 M Bis-Tris, pH 6.5, was used for X-ray diffraction experiments. Crystals appeared as needles after one day of equilibration at 20° C. and reached 100 μm in their longest dimension on Day 30. These needles were used to macro seed into 2 μl hanging drop vapor diffusion plates with drops containing equal volumes of 10 mg/ml Nb82 and reservoir conditions optimized around the original crystallization condition varying the concentrations of PEG and ammonium sulfate. New crystals appeared in 2% (w/v) PEG MME 550, 1.5-1.8 M ammonium sulfate, 0.1 M Bis-Tris, pH 6.5, on Day 5. The crystals grew into cubes with largest samples being 125 μm in their longest dimension and were harvested on Day 30 post-seeding from the mother liquor mixed with 1 M lithium sulfate as the cryo-protectant into Hampton Research loops and plunge-frozen into liquid nitrogen.

Data Collection and Structure Refinement

X-ray diffraction data of the Nb82 crystal were collected at 100 K at the NSLS II 17-ID-1 beamline equipped with a DECTRIS Eiger 9M detector. Data were processed with fastdp and XDS and scaled using XSCALE. Initial phases were obtained by molecular replacement using a nanobody structure as search model (PDB ID 5LMJ) with the CCP4 program PHASER. Initial models were improved with multiple rounds of rebuilding using Coot and refinement using REFMAC version 5.8. The quality of the model was assessed with Coot validation tools and the wwPDB validation servers. Statistics are shown in Table 3. The final model contains 4 Nb82 molecules in the asymmetric unit.

TABLE 3
Data collection and refinement statistics

| Data collection | Nb82 | |
|---|---|---|
| Space group | C2221 | |
| Cell dimensions | | |
| a, b, c (Å) | 77.7, 82.4, 170.7 | |
| α, β, γ (°) | 90.00, 90.00, 90.00 | |
| Resolution (Å)a | 29.67-2.0 | (2.052-2.0) |
| Rmerge | 0.07 | (0.39) |
| Rpim | 0.030 | (0.164) |
| CC1/2 | 0.999 | (0.985) |
| [symbol] I/σ(I) [symbol] | 3.52 | (2.00) |
| Completeness (%) | 99.9 | (99.9) |
| Total reflections | 249,354 | (2,845) |
| Unique reflections | 37,476 | (473) |
| Refinement | | |
| Rwork/Rfree | 0.19/0.24 | (0.22/0.28) |

| | |
|---|---|
| No. of atoms | |
| Protein | 4331 |
| Ligand/ion | — |
| Water | 278 |
| B-factors (Å2) | |
| Protein | 36.0 |
| Water | 44.6 |
| R.m.s. deviations | |
| Bond lengths (Å) | 0.01 |
| Bond angles (°) | 1.57 |
| Ramachandran plot | |
| Favored (%) | 97.8 |
| Allowed (%) | 1.5 |
| Disallowed (%) | 0.5 |

aValues for the outer shell are given in parentheses

Nb-Mediated Shift of Naᵥs by Sizing Exclusion Chromatography.

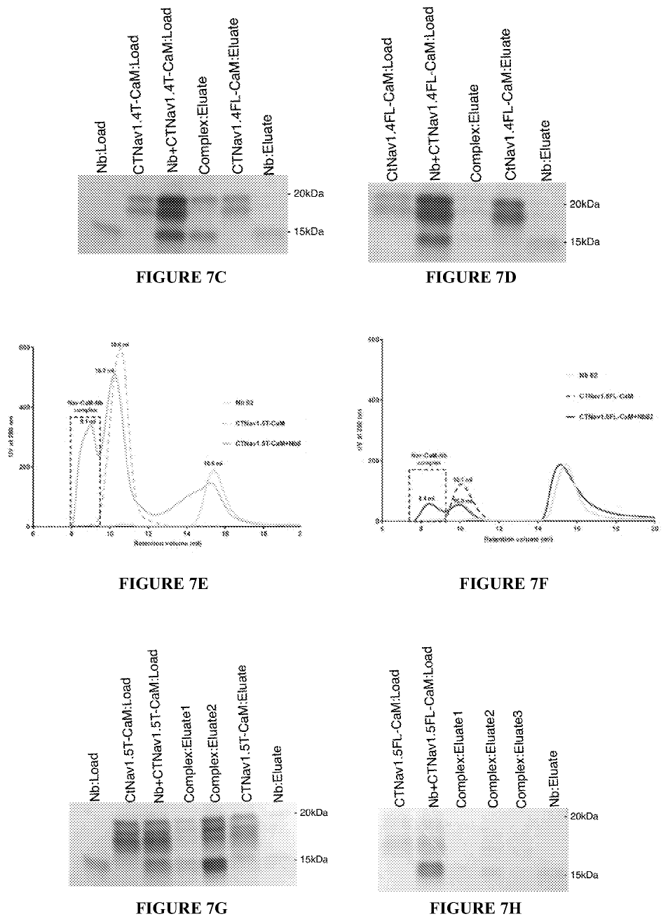

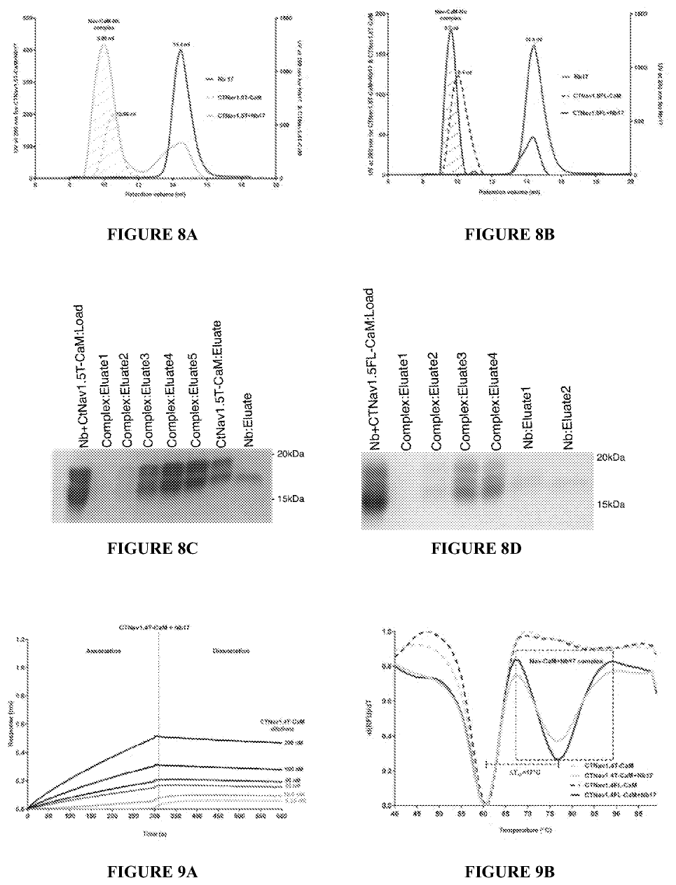

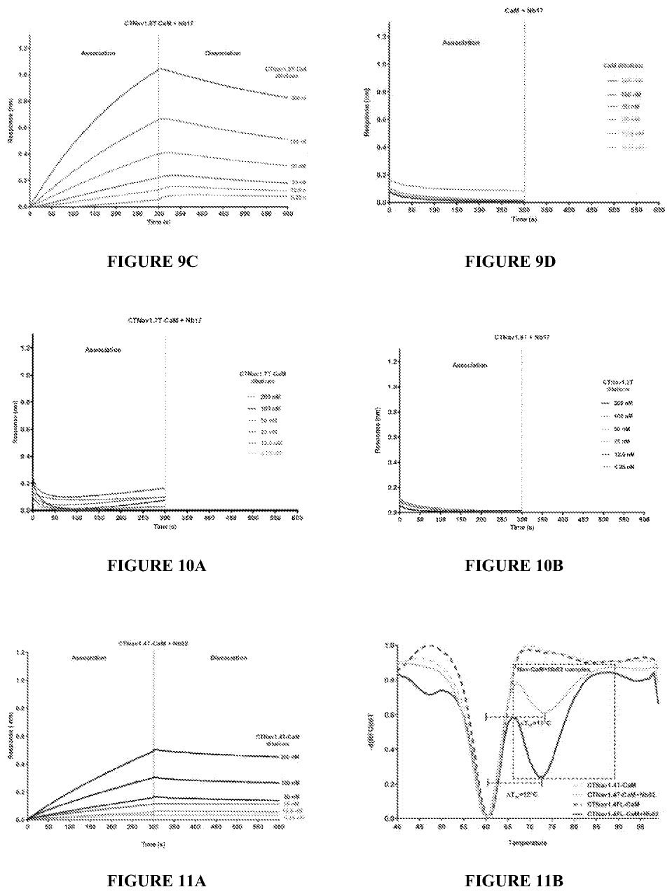

Mobility in gel filtration chromatography was performed to verify formation of CTNaᵥ-CaM+Nb complexes using purified proteins (FIGS. 7 and 8). Nbs and Naᵥs were mixed in a 1.2:1 molar ratio, incubated for 2 h to overnight at 4° C. and run on a Superdex 75 10/300 GF column (GE) using 20 mM Tris, 50 mM NaCl, pH 7.4. Chromatograms were exported as Excel files to GraphPad prism for analysis and plotting.

Nb17 and Nb82 Binding Kinetics (BLI)

Nb17 and Nb82 binding to Naᵥ1.4 and Naᵥ1.5 was measured by Bio-Layer-Interferometry (BLI) using the Octet RED96 instrument (ForteBIO, Pall Corp., US). Data were acquired in the kinetics mode using 200 μl protein/well in a 96-well plate format. Data were analyzed using the Data acquisition software v9.0 and Data analysis software v9.0 respectively (ForteBIO, Pall Corp., USA). His-tagged proteins Nb17 and Nb82 or just buffer was immobilized on Ni-NTA biosensors. Nb17 and Nb82 loading was done at 2.5 μg/ml concentration for 300 s to prevent overcrowding and self-association of the ligand. CTNaᵥ1.4T-CaM, CTNaᵥ1.5T-CaM, CTNaᵥ1.7T-CaM and CTNaᵥ1.9T were tested as analytes. To measure Nb association with Naᵥ proteins, the Nb loaded sensors were first transferred to wells containing blocking reagent (0.1% biocytine) in assay buffer for 150 s to prevent non-specific binding and then to wells containing assay buffer until a stable baseline was reached (100 s). Following this, sensors were dipped into wells with 1:2 serially diluted Nav proteins (at 200, 100, 50, 25, 12.5, and 6.25 nM concentrations) for 300 s followed by a 300 s dissociation step in assay buffer. All experiments were carried out at 25° C. and acquisition standard at 5 Hz with the assay plate shaking at 2000 rpm.

Thermostability Assay of CTNaᵥs+Nb Complexes Using Differential Scanning Fluorimetry (DSF)

Nbs (Nb17, Nb82) and CTNaᵥ1.4-CaM+Nb complexes (CTNaᵥ1.4T-CaM and CTNaᵥ1.4FL-CaM) and only CaM were thermally denatured in the presence of SYPRO Orange dye (1000× stock, Life Technologies) and their stability was tested by ThermoFluor T_{M} assay using DSF (BioRad). All proteins for the assay were used at 6 and 13 μM concentrations in PBS. Protein samples were divided into triplicates of 20 μl reactions each with 50× concentration of the dye and transferred to a thin-wall 96-well PCR plate (BioRad) and sealed using an opti-seal cover (BioRad) and centrifuged to spin down the samples at 2500 g for 2 min. Fluorescence intensity was measured using the 96-well SYPRO Orange template on BioRad CFX machine with a temperature ramp of 1° C./min with 10 s hold/° C. Melting curves obtained were exported as Excel files, normalized and baseline-corrected for control experiments (CaM+Nb curves) and analyzed by Graph prism software (Prism 6.0 v6.07) and plotted as dF in arbitrary units along the Y-axis and temperature (° C.) along the X-axis. The peak temperature values obtained were used as the Tₘ of the protein sample and the shifts in Tₘ were used to compare the stability of the protein complexes.

Western Blots Using Nb82 to Detect Endogenous, Overexpressed and Purified Naᵥ1.4 and Naᵥ1.5.

Western blot analysis was performed to assess whether Nb82 detect Naᵥ1.4 from skeletal muscle and Naᵥ1.5 from cardiac tissue as well as Naᵥ1.5 wt IPSC differentiated cardiomyocytes cells (CM), HEK293 transiently transfected with Naᵥ1.5. Tissue samples were sonicated in PBS and centrifuge at 10,000 g for 30′. The supernatants were loaded and run on any Kd (MiniProtean) SDS-PAGE gel. Proteins were transferred to an PVDF membrane for on an iBLOTT 2 P3 at 20 mV, blocked for 1 hour while shaker in 1×PBST with 5% milk, washed 5× in PBST. Protein were detected with Nb-His and visualized using anti-HIS conjugated HRP (SIGMA, catalog, 1:200) or using a pan-Nav antibody (SIGMA, catalog, 1:200) and visualized using an anti-mouse IGG HRP.

Western blots using Nb82 to detect purified CTNaᵥ-CaM complexes. Western blot analysis was performed to assess whether Nb82 can detect CTNaᵥ1.4-CaM and CTNaᵥ1.5-CaM. ˜1 μg/lane of purified CTNaᵥ1.4T-CaM, CTNaᵥ1.4FL-CaM, CTNaᵥ1.5T-CaM, CTNaᵥ1.5FL-CaM, CTNaᵥ1.7T-CaM, CTNaᵥ1.7FL-CaM, CTNaᵥ1.9T, CTNaᵥ1.9FL, CaM, Nb87, Nb17 were run on any Kd (MiniProtean) SDS-PAGE gel. Proteins were transferred and develop as described above.

### Example 2

### Generation and Selection of NAᵥ1.4-Specific NBS

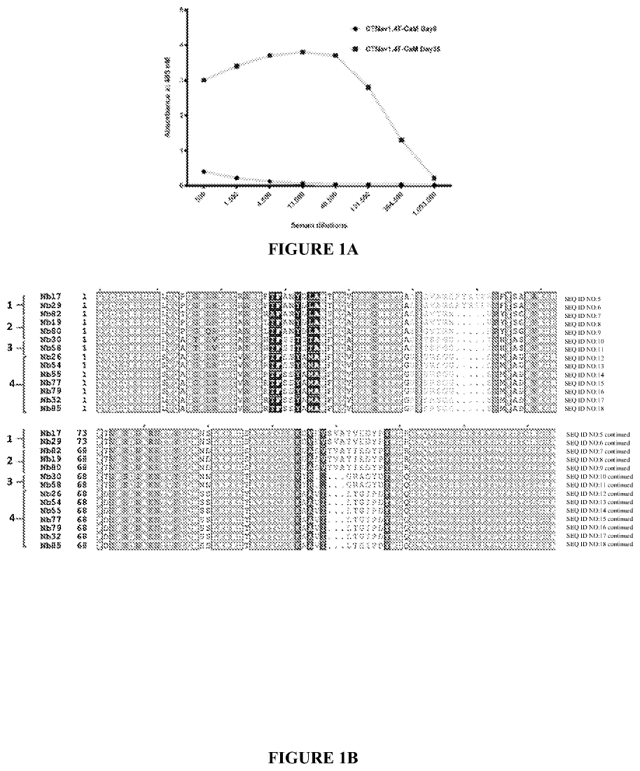

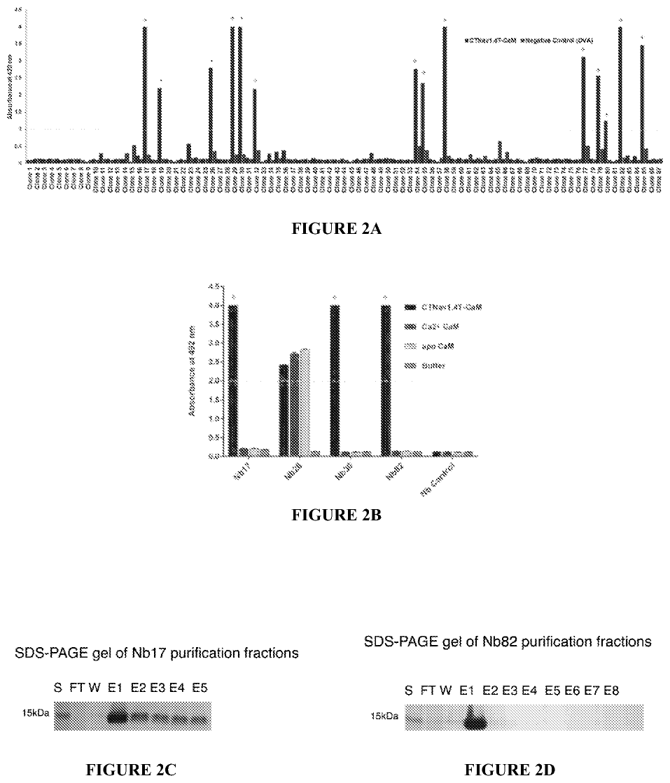

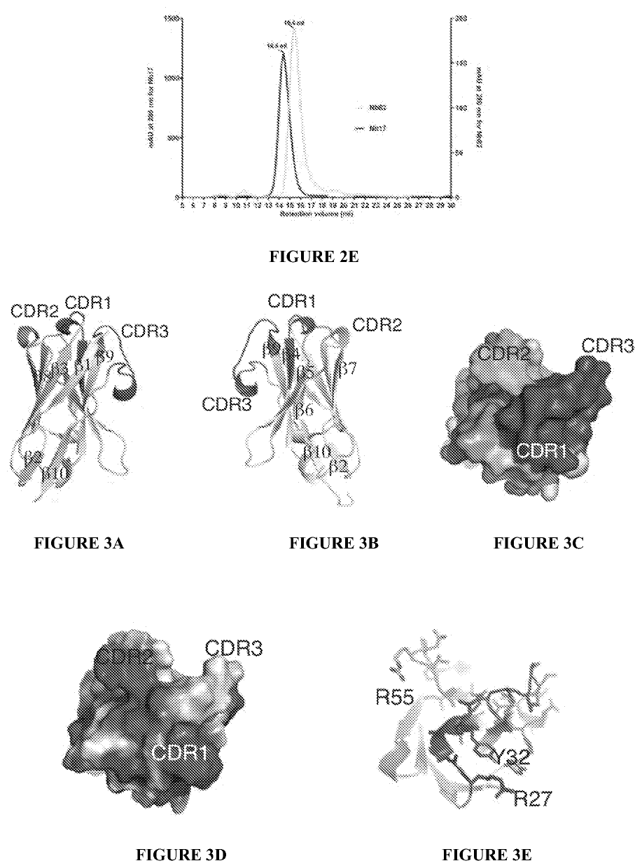

GST-tagged truncated (T), C-terminal (CT) of Naᵥ1.4 encompassing residues 1694-1764 (CTNaᵥ1.4T) were expressed and purified in complex with calmodulin (CTNaᵥ1.4T-CaM) from bacterial cells. To generate Nbs specific for folded CTNaᵥ1.4T-CaM, two llamas were immunized three times with this complex and their humoral immune response was evaluated by ELISA (FIG. 1A). Two Nb phage display libraries were generated with size 5.6×10⁷ and 2.1×10⁸ clones (llama #1 and #2, respectively) and Nay-specific Nbs were selected after two rounds of panning A total of 87 randomly chosen clones were expressed and tested by ELISA (FIG. 2). Amongst them, 14 clones were specific against Naᵥ1.4 (FIG. 1B, and Table 4). These results allowed to classify their sequences in four different families based on the length and variability of their CDR3 and CDR2 regions.

TABLE 4
Sequences of the nanobodies obtained in the 14 clones

| SEQ ID NO | Nanobody name | Sequence |
|---|---|---|
| SEQ ID NO: 5 | Nb17 | QVQLQESGGGLVQPGGSLRLSCRASGFTFANYDLAWTR |
| | | QVPGKEREFVASIGVARGRTRTPATFYSASAKGRFTVSR |
| | | DDARNTVYLQMNSLKPEDSAVYYCAAKDVEVSVATVE |
| | | DYPYWGRGTQVTVSSGRYPYDVPDYGSGRA |
| SEQ ID NO: 6 | Nb19 | QVQLQESGGGLVQPGGSLRLSCAASGLTFANYDLAWSR |
| | | QAPGKQREFVASIGVTRNPPYYSGSVKGRFTVSRDNAKE |
| | | TVYLQMNDLKPEDSAVYYCAAKDASVTVATIEDYPYW |
| | | GRGTQVTVSSGRYPYDVPDYGSGRA |
| SEQ ID NO: 7 | Nb26 | QVQLQESGGGSVQAGGSLRLSCVASGRTFSSYAMAWF |
| | | RQVPGKEREFVGRISRSGGSTMYADSVKGRFDISRDNAK |
| | | NTVFLQMSSLKPEDTAVYYCAAKAAVILTGIPDYWGQ |
| | | GTQVTVSSGRYPYDVPDYGSGRA |
| SEQ ID NO: 8 | Nb29 | QVQLQESGGGLVQPGGSLKLSCRASGFTFANYDLAWTR |
| | | QVPGKEREFVASIGVARGRTRTPATFYSASVKGRFTVSR |
| | | DDARNTVYLQMNSLKPEDSAVYYCAAKDVEVSVATVE |
| | | DYPYWGRGTQVTVSSGRYPYDVPDYGSGRA |
| SEQ ID NO: 9 | Nb30 | QVQLQESGGGLVQAGTSLVLSCAISGHTFSITGTAWFR |
| | | QAPGKEREFVAGLTSSGDITHYASSVKGRFTISSDIAKNT |
| | | VYLQMNNLKPEDTAVYYCTSVIRGRAGYDTWGQGTQ |
| | | VTVSSGRYPYDVPDYGSGRA |
| SEQ ID NO: 10 | Nb32 | QVQLQESGGGLVQAGGSLRLSCVASGRTFSSYAMAWF |
| | | RQVPGKEREFVGRISRSGGSTMYADSVKGRFDISRDNAK |
| | | NTVFLQMSSLKPEDTAVYYCAAKAAVILTGIPDYWGQ |
| | | GTQVTVSSGRYPYDVPDYGSGRA |
| SEQ ID NO: 11 | Nb54 | QVQLQESGGGSVQAGGSLRLSCVASGRTFSSYAMAWF |
| | | RQVPGKEREFVGRISRSGGSTMYADSVKGRFDISRDNAK |
| | | NTVFLQMSSLKPEDTAVYYCAAKAAVILTGIPDYWGQ |
| | | GTQVTVSSGRYPYDVPDYGSGRA |
| SEQ ID NO: 12 | Nb55 | QVQLQESGGGSVQAGGSLRLSCVASGRTFSSYAMAWF |
| | | RQVPGKEREFVGRISRSGGSTMYADSVKGRFDISRDNAK |
| | | NTVFLQMSSLKPEDTAVYYCAAKAAVILTGIPDYWGQ |
| | | GTQVTVSSGRYPYDVPDYGSGRA |
| SEQ ID NO: 13 | Nb58 | QVQLQESGGGLVQAGTSLVLSCAISGHTFSITGTAWFR |
| | | QAPGKEREFVAGLTSSGDITHYASSVKGRFTISSDIAKNT |
| | | VYLQMNNLKPEDTAVYYCTSVIRGRAGYDTWGQGTQ |
| | | VTVSSGRYPYDVPDYGSGRA |
| SEQ ID NO: 14 | Nb77 | QVQLQESGGGSVQAGGSLRLSCVASGRTFSSYAMAWF |
| | | RQVPGKEREFVGRISRSGGSTMYADSVKGRFDISRDNAK |
| | | NTVFLQMSSLKPEDTAVYYCAAKAAVILTGIPDYWGQ |
| | | GTQVTVSSGRYPYDVPDYGSGRA |
| SEQ ID NO: 15 | Nb79 | QVQLQESGGGSVQAGGSLRLSCVASGRTFSSYAMAWF |
| | | RQVPGKEREFVGRISRSGGSTMYADSVKGRFDISRDNAK |
| | | NTVFLQMSSLKPEDTAVYYCAAKAAVILTGIPDYWGQ |
| | | GTQVTVSSGRYPYDVPDYGSGRA |
| SEQ ID NO: 16 | Nb80 | QVQLQESGGGLVQPGGSQRLSCAASGLTFANYDLAWS |
| | | RQAPGKQREFVASIGVTRNPPYYSGSVKGRFTVSRDNAK |
| | | ETVYLQMNDLKPEDSAVYYCAAKDASVTVATIEDYPY |
| | | WGRGTQVTVSSGRYPYDVPDYGSGRA |
| SEQ ID NO: 17 | Nb82 | QVQLQESGGGLVQTGGSLRLSCKASGRAFARYDLAWS |
| | | RQAPGKQREFVASIGVTRNPPYYSGSVKGRFTVSRDNAK |
| | | ETVYLQMNDLKPEDSAVYYCAAKDASVTVATIEDYPY |
| | | WGRGTQVTVSSGRYPYDVPDYGSGRA |
| SEQ ID NO: 18 | Nb85 | QVQLQESGGGLVQAGGSLRLSCVASGRTFSSYAMAWF |
| | | RQVPGKEREFVGRISRSGGSTMYADSVKGRFDISRDNAK |
| | | NTVFLQMSSLKPEDTAVYYCAAKAAVILTGIPDYWGQ |
| | | GTQVTVSSGRYPYDVPDYGSGRA |

Bold: CDR1
underlined: CDR2
bold underlined: CDR3

Family1 and family2 have two and three VHH clones, respectively, all of which display a 15-aa length CDR3 but vary in the length of CDR2 (Family1, 13 aa; Family2, 8 aa). The two VHH clones of family3 have shorter CDR3s (10 aa). Family4 comprises 7 VHH clones with a 12-aa long CDR3 and 8-aa long CDR2 (FIG. 1B). One representative clone from each family was selected and screened by ELISA for specificity to purified CTNaᵥ1.4T-CaM, Ca2+CaM and apoCaM (Nb17, 26, 30 and 82, FIG. 1B). Nb17, Nb30 and Nb82 specific to CTNaᵥ1.4T-CaM. Nb26 was eliminated from further studies because it recognized not only CTNaᵥ1.4T-CaM but also CaM by itself in periplasmic ELISA (FIG. 2B). The other three clones were targeted for expression and purification in E. coli for further biophysical and biochemical characterization.

TABLE 5
Sequences of the CDRs in the 4 families

| SEQ ID NO | Name | Sequence |
|---|---|---|
| SEQ ID NO: 19 | CDR1_1 | FTFANYDLA |
| SEQ ID NO: 20 | CDR2_1 | SIGVARGRTRTPA |
| SEQ ID NO: 21 | CDR3_1 | KDVEVSVATVEDYPY |
| SEQ ID NO: 22 | CDR1_2.1 | LTFANYDLA |
| SEQ ID NO: 23 | CDR1_2.2 | RAFARYDLA |
| SEQ ID NO: 24 | CDR2_2 | SIGVTRNP |
| SEQ ID NO: 25 | CDR3_2 | KDASVTVATIEDYPY |
| SEQ ID NO: 26 | CDR1_3 | HTFSITGTA |
| SEQ ID NO: 27 | CDR2_3 | GLTSSGDI |
| SEQ ID NO: 28 | CDR3_3 | VIRGRAGYDT |
| SEQ ID NO: 29 | CDR1_4 | RTFSSYAMA |
| SEQ ID NO: 30 | CDR2_4 | RISRSGGS |
| SEQ ID NO: 31 | CDR3_4 | KAAVILTGIPDY |

Nb17, Nb30 and Nb82 were sub-cloned into a pHEN6-His vector as a C-terminal 6×-His tagged fusion protein and successfully expressed in the periplasm of E. coli BL21(DE3) Rosetta gami-2 cells. The C-terminal 6×-His tagged Nbs were extracted from the E. coli periplasm using a combination of thermal and osmotic shock methods 25. Nb30 was not pursued further due to low expression levels. Nb17 and Nb82 were purified via Ni-NTA chromatography, followed by size exclusion chromatography (FIGS. 2C, 2D and 2E). Both Nbs behaved as monomers in solution and were detected as a single, homogenous peak on size exclusion chromatography with retention volumes of 14.4 and 15.6 ml, respectively, on a Superdex 75 10/300 GF column (FIG. 2E). These volumes were in line with their respective molecular weights of 17.4 and 16.9 kDa. Nb17 and Nb82 had a theoretical pI of ˜8.8 and ˜8.5, respectively.

### Example 3

### NB82 has an Extended CDR3 Loop

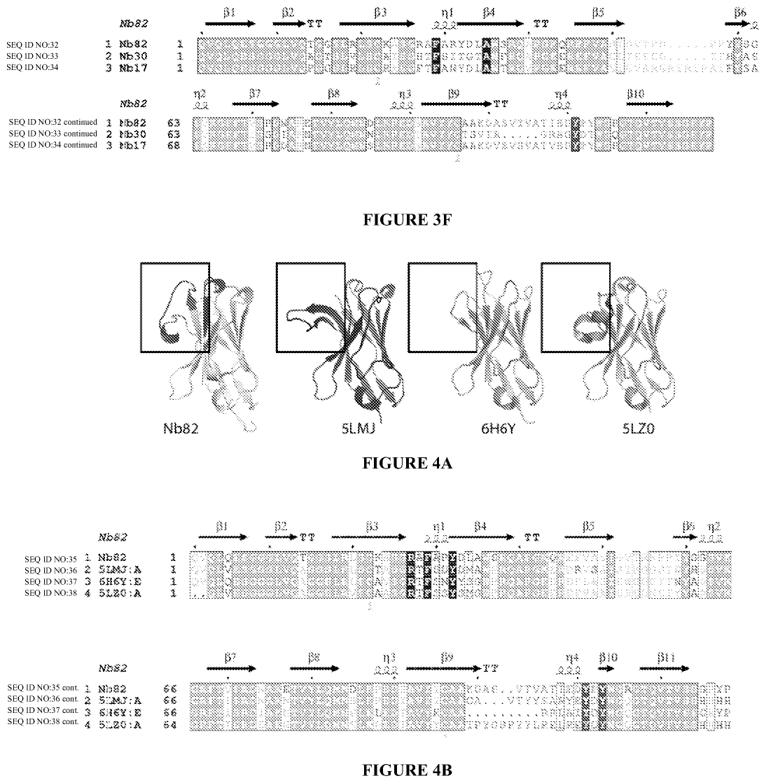

The structure of Nb82 was determined by X-ray crystallography to 2.0 Å resolution. The structure was refined to a R_{work}/R_{free} of 0.19/0.24 with excellent geometry (Table 3, FIG. 3). The asymmetric unit includes four copies of Nb82 with almost identical conformations (C^{a} RMSD <0.17 Å). Nb82 bears the classical immunoglobulin fold with two β-sheets of four antiparallel β-strands (β1-β3-β8-β7 and β6-β5-β4-β9) and a smaller β-sheet made up of 2 parallel β-strands, β2 and β10, that elongate the β6-β5-β4-β9 β-sheet (FIGS. 3A and 3B). The structure displays good electron density for all three variables, epitope recognizing CDR regions. The fold is decorated by two π-helices present in CDR1 and CDR3 formed between the β3-β4 and β9-β10 loops. π-Helices have been observed in other Nbs. CDR3, the usual major contributor for antigen recognition and specificity, folds as a random coil that wraps around the β6-β5-β4-β9 β-sheet finishing up in a π-helix of seven residues. Comparison of the sequence of Nb82 with Nb17 and Nb30 (FIG. 3F) and its structure with other Nbs (PDB IDs 5LMJ, 6H6Y, and 5LZ⁰) highlighted the differences in the fold of their CDR3. Specifically, the CDR3 of Nb82 was not only the most divergent in length but also in conformation (FIGS. 4A and 4B). As part of the paratope CDR1 and CDR2 formed a positive charged surface (FIG. 3C).

### Example 4

### NB17 and NB82 are Specific for the CTNAᵥ1.4 and 1.5 Isoforms

To evaluate whether the selected Nbs were pan-Naᵥs or isoform specific, ELISAs were performed (FIG. 5A) with the purified Nbs and each of 4 purified CTNav isoforms [truncated (T) or full-length versions (FL)] in complex with CaM (8 different CTNaᵥ-CaM in total) (FIG. 5B). Interestingly, Nb82 and Nb17 recognized both the T and FL CTNaᵥ1.4-CaM (skeletal) and CTNaᵥ1.5-CaM (cardiac) but not CTNaᵥ1.7-CaM nor CTNaᵥ1.9-CaM (FIG. 5A). Also, these Nbs did not cross-react with free CaM nor GST (FIGS. 5 and 6).

The complexes including the Nbs were analyzed by size exclusion chromatography (FIG. 7). The CTNaᵥ1.4T-CaM+Nb82 (FIGS. 7A and 7C) and CTNaᵥ1.4FL-CaM+Nb82 complexes (FIGS. 7B and 7D) eluted about 2 mL earlier than the equivalent complexes without the Nb. SDS-PAGE analysis of the elution fractions showed that the new peaks contained all 3 proteins; CTNaᵥ1.4, CaM and the Nb (FIGS. 7C and 7D).

CTNaᵥ1.5 showed a similar behavior. The complexes CTNaᵥ1.5T-CaM+Nb82 and CTNaᵥ1.5FL-CaM+Nb82 eluted 1.0 and 1.5 ml before the CTNaᵥ1.5T-CaM complex, respectively (FIGS. 7E and 7F). SDS-PAGE confirmed the presence of the three proteins (CTNaᵥ1.5, CaM and Nb) in the fractions containing the complexes (FIGS. 7G and 7H).

Interestingly, neither the ratio of CTNaᵥ-CaM to Nb82 nor the temperature nor the incubation time resulted in a 100% complex formation as detected by gel filtration. Since the hydrodynamic radii of complexes correlated with the length of the CTNaᵥ used, it suggested that the extra −50 residues of the CTNaᵥFL-CaMs (FIG. 5B) did not fold back onto the shorter CTNaᵥ1.4T-CaM and CTNaᵥ1.5T-CaM (FIG. 7) allowing both truncated and full length CTNaᵥ-CaM to share the epitope specificity of Nb82.

Nb17 harbored a more puzzling paratope. Although ELISA tests showed that it recognized CTNaᵥ1.5-CaM, the elution profile of the CTNaᵥ1.5-CaM+Nb17 did not show a fully resolved new peak indicative of the complex. Instead, the CTNaᵥ1.5T-CaM+Nb17 (FIGS. 8A and 8C) and CTNaᵥ1.5FL-CaM+Nb17 (FIGS. 8B and 8D) peaks elute 0.5 ml before the CTNaᵥs without the Nb followed by an asymmetric Nb17 peak, suggesting that Nb17 and Nb82 recognized different epitopes on CTNaᵥ1.5-CaM since they affected the hydrodynamic radius differently.

### Example 5

### Nanobodies Bind with Nanomolar Affinities to CTNAᵥ

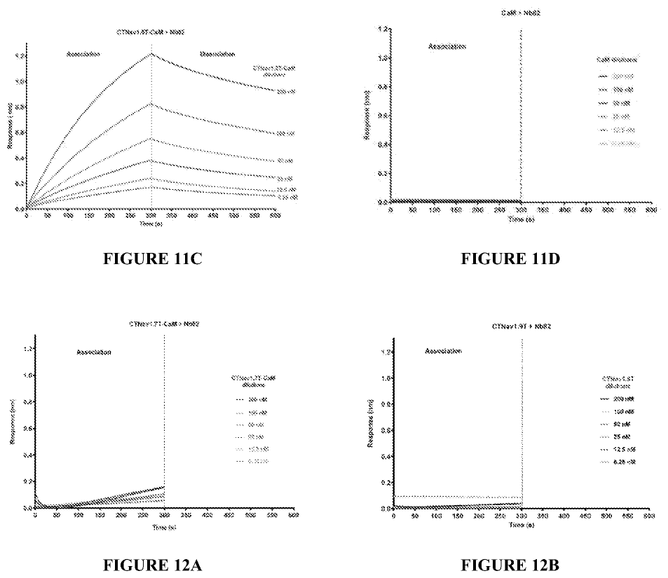

The CTNaᵥ1.4-CaM+Nb and CTNaᵥ1.5-CaM+Nb (CTNaᵥ1.4(5)-CaM+Nb) complexes were further characterized by i) determining the kinetic parameters of this interaction using Bio-Layer-Interferometry (BLI) and ii) studying their thermal stability by differential scanning fluorimetry (DSF) (FIGS. 9 and 11). Binding isotherms were generated in BLI experiments using Nb17 or Nb82 coated Ni-NTA biosensors and CTNaᵥ1.4(5)T-CaM, CTNaᵥ1.7T-CaM, CTNaᵥ1.9T and CaM proteins as analytes on an Octect RED 96 instrument (Forte Bio, Pall Life Sciences). The change in resonance units (RU) with time was recorded at different concentrations of purified CTNaᵥT-CaM proteins or CaM alone showing clear 1:1 binding of the Nbs to CTNaᵥ1.4(5)T-Ca M proteins reaching steady state in 300 s.

With Nb17, analysis of the dose-responses of association and dissociation curves (FIGS. 9A and 9C) indicated that it bound to CTNaᵥ1.4T-CaM and CTNaᵥ1.5T-CaM with dissociation constants (K_{Ds}) of 41.1 and 60.5 nM respectively (Table 6). However, no binding was observed when CaM alone was used as analyte (FIG. 9D): it displayed a non-association/no-binding curve. Further, DSF measurements demonstrated that Nb17 robustly stabilizes the CTNaᵥ1.4-CaM+Nb complex as observed by an increase in the melting temperature (T_{M}) of CTNaᵥ1.4T-CaM and CTNaᵥ1.4FL-CaM, corresponding to a shift (ATM) of 17° C. (FIG. 9B). Similarly, BLI experiments using Nb82 showed that it bound to CTNaᵥ1.4T-CaM and CTNaᵥ1.5T-CaM with 50.2 and 63.2 nM affinity (FIGS. 11A and 11C) but not to CaM alone (FIG. 11D; Table 5. Further, Nb82 stabilized the CTNaᵥ1.4-CaM complexes (higher Tₘ) of CTNaᵥ1.4T-CaM and CTNaᵥ1.4FL-CaM, displaying thermal shifts of 13 and 12° C. respectively (FIG. 11B).

TABLE 6
Kinetic and binding parameters determined
by BLI for muscle-isoforms CTNaᵥ1.4T-CaM and
CTNaᵥ15T-CaM titrated with Nb17 and Nb82

| Nb | Naᵥ proteins | | | |
|---|---|---|---|---|
| (ligand) | (analyte) | K_{D} (M) | kₒₙ (M⁻¹s⁻¹) | k_{dis} (1/s) |
| Nb17 | CTNaᵥ1.4T-CaM | 4.11E⁻⁰⁸ ± | 1.80E⁻⁰⁴ ± | 7.39E⁻⁰⁴ ± |
| | | 9.89E⁻¹⁰ | 3.50E⁻⁰² | 1.05E⁻⁰⁵ |
| | CTNaᵥ15T-CaM | 6.05E⁻⁰⁸ ± | 1.78E⁻⁰⁴ ± | 1.07E⁻⁰³ ± |
| | | 5.80E⁻¹⁰ | 1.51E⁻⁰² | 4.74E⁻⁰⁶ |
| Nb82 | CTNaᵥ1.4T-CaM | 5.02E⁻⁰⁸ ± | 8.98E⁻⁰³ ± | 4.51E⁻⁰⁴ ± |
| | | 8.87E⁻¹⁰ | 1.32E⁻⁰² | 4.41E⁻⁰⁶ |
| | CTNaᵥ15T-CaM | 6.32E⁻⁰⁸ ± | 1.78E⁻⁰⁴ ± | 1.13E⁻⁰³ ± |
| | | 6.75E⁻¹⁰ | 1.71E⁻⁰² | 5.28E⁻⁰⁶ |

### Example 6

### NB82 Detect NAᵥ1.4 and NAᵥ1.5 from Tissues

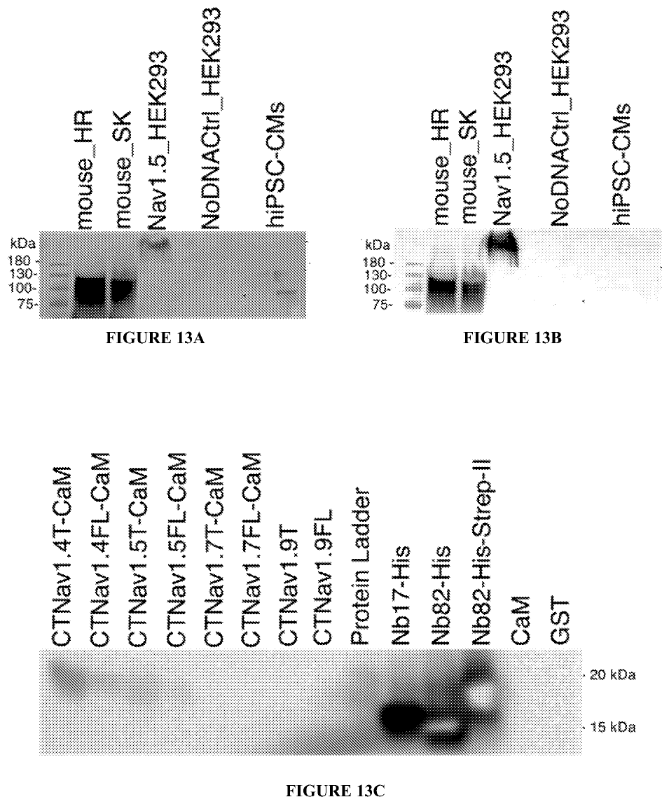

Western blot analysis using Nb82-His detected full-length channels: Naᵥ1.4 from skeletal muscle, Naᵥ1.5 from cardiac tissue, Naᵥ1.5 wt IPSC differentiated cardiomyocytes cells (CM), and Naᵥ1.5-HEK293 (FIG. 1.3A). Furthermore, comparison with WB detected with an anti-pan-Naᵥ antibody displayed bands at equivalent molecular weight. Interestingly, Nb82 was found more sensitive at detecting the Naᵥ1.5 from IPSC since the band was undetected by anti-pan-Naᵥ antibody (FIG. 13B). In agreement with the results from ELISA, western blot analysis showed that Nb82 specifically detected CTNaᵥ1.4-CaM, CTNaᵥ1,4FL-CaM, CTNaᵥ1.5T-CaM, CTNaᵥ1.5FL-CaM amongst all other purified CTNaᵥ-CaM proteins (FIG. 13C).

Fusion of a Nanobody with an Active E3 Ligase Domain Removes Naᵥ1.5 from the Plasma Membrane

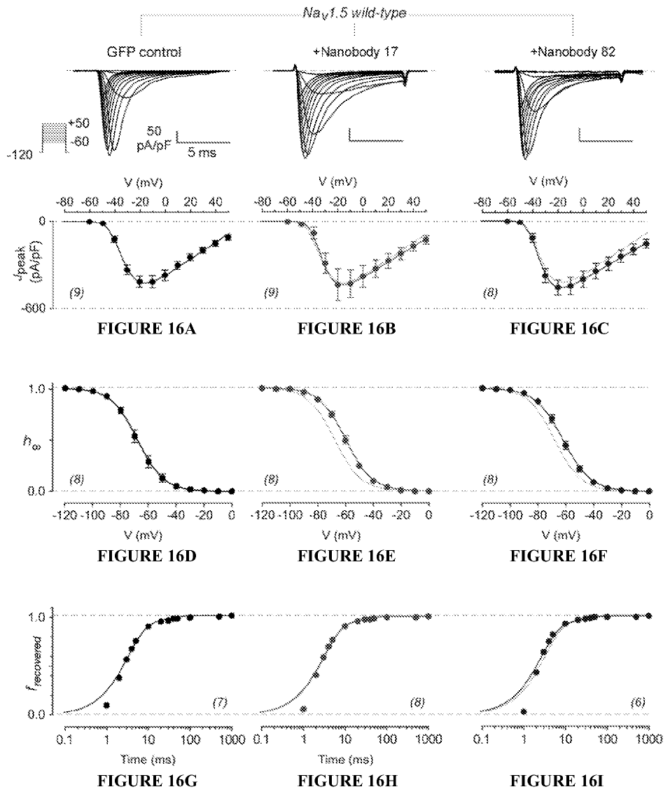

The effect of each nanobody on the activity of the channel was assessed by transfecting HEK293 cells with either Nb17 or Nb82. The function of Naᵥ1.5 channel was assessed as elicited in response to a family of voltage steps from −60 to +50 mV and evaluated the average peak density (FIG. 16). The comparison of cells transfected with Naᵥ1.5 vs the cells co-transfected with Naᵥ1.5 and either Nb17 or Nb82 display that the nanobodies are silent.

The DNA that code for the nanobodies Nb17 and Nb82 was used to fuse to the DNA sequence that codes for NEDD4L_HECT domain (residues 60-975) to deliver an active E3 ligase that regulates proteostasis of Naᵥ1.5 and called it NanoMaN,

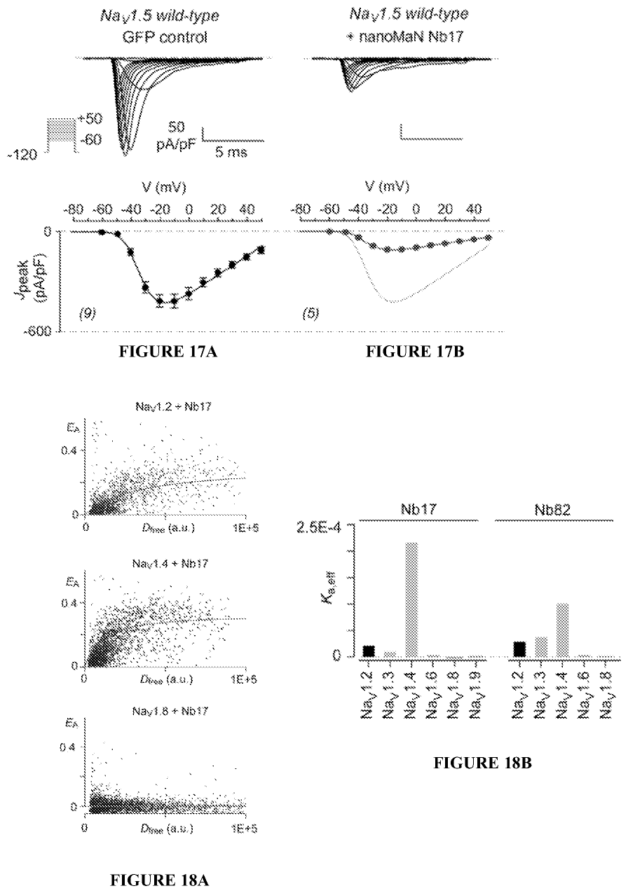

HEK293 cells transfected with NanoMaNs were used to undertake whole-cell and single-channel electrophysiology analysis to quantify changes in steady-state and late Ina current (FIG. 17). Interestingly, using a NanoMan where. Nb17 as the carrier of the cargo E3Ligase, displays a strong reduction of Ina and infer a reduction of mature ion channels at the plasma membrane,

### Example 7

### Discussion

Two anti-CTNaᵥ Nbs (Nb17 and Nb82) that selectively bind to CTNaᵥ1.4-CaM (skeletal muscle) and CTNaᵥ1.5-CaM (cardiac muscle) but not to CaM alone nor to other isoforms such as CTNaᵥ1.7 and CTNaᵥ1.9 were expressed and purified. The crystal structure of Nb82 reveals the expected immunoglobulin fold with two β-sheets of four and five antiparallel—β strands. CDR3, the major paratope contributor of Nb82 was particularly long (15 aa) for a llama derived Nb and formed a positively charged surface with CDR1 and CDR2. Further, BLI kinetic experiments revealed that CTNaᵥ1.4(5)-CaM isoforms bind to Nb17 and Nb82 with nanomolar affinity (FIGS. 9 and 11). In addition, Nb17 and Nb82 were found soluble, stable and thermally stabilized CTNaᵥ1.4-CaM by robustly increasing its melting temperature (FIG. 9B). Given their outstanding properties, Nb17 and Nb82 show great potential as molecular visualization probes to study Nay-channels in cells and as potential Naᵥ-channel modulators.

Nanobodies are single antibody domain proteins obtained from the heavy chain only antibodies that are part of the immune response of camelids. Despite being a single domain (VHH), nanobodies have specificity and affinity comparable, and sometimes greater than conventional antibodies. Their smaller size and sometimes long CDR3 endows them with advantages such as accessibility to cryptic/hidden epitopes and improved tissue penetration.

Two Nbs with nanomolar affinity for CT-Naᵥ1.4 and CT-Naᵥ1.5were raised, selected and characterized. These Nbs, selected from a very large phage display library, are specific for the C-termini of Naᵥ1.4 and Naᵥ1.5 and do not recognize other isoforms such as Naᵥ1.7 and Naᵥ1.9 or CaM by itself, as determined in ELISA experiments. Complex formation, as measured by size exclusion chromatography experiments, suggest that they show high affinity for CT-Naᵥ1.4 and CT-Naᵥ1.5 and that both Nbs recognize different epitopes. This offers a potential to simultaneously block and or activate different regions of the Naᵥ channels on the membrane with high specificity.

Since they can be expressed in the cytosol, Nbs are attractive tools for targeting Naᵥ proteins. Also, ease of humanization of llama-Nbs, low off-target effects and high isoform selectivity, no risk of metabolic toxicity and the availability of transfection carriers such as viruses for delivery, make them superior biologicals for targeted therapy.

### Example 8

### Flow Cytometric Fret 2-Hybrid Assay

To determine whether Nb17 and Nb82 interact with holo-Naᵥ1.5 channels in live cells, a flow-cytometry based FRET 2-hybrid assay was utilized (Rivas et al., Methods in Enzymology. 653, 2021.). FRET (fluorescence resonance energy transfer or Förster resonance energy transfer) between fluorescent proteins is a non-invasive technique available to detect direct protein-protein interactions in living cells. It is based upon the energy transfer from an excited donor fluorophore to an adjacent acceptor fluorophore, resulting in decreased fluorescence emission by the donor and enhanced fluorescence emission by the acceptor.

A flow cytometric FRET 2-hybrid assay for detecting nanobody interaction with Naᵥ1.5 was performed as in previous studies. Briefly, HEK293 cells were cultured in 12 well plates and transfected with polyethylenimine (PEI) 25 kDa linear polymer (Polysciences #2396602). For each experiment, the following were co-transfected: 0.5 μg of Nb17 or Nb82-fused to Cerulean; 2 μg of Venus tagged Naᵥ1.5; and 0.5 μg of t-Antigen. The cDNA pairs were mixed together in 200 μl of serum-free DMEM media and 5 μl of PEI was added into each sterile tube. Following 15 minutes of incubation, PEI/cDNA mixtures were added to the 12 well plates and cells were cultured for 2 days prior to experimentation. Protein synthesis inhibitor cycloheximide (100 μM) was added to cells 2 h prior to experimentation to enhance fluorophore maturation. For FRET measurements, we utilized an LSR II (BD Biosciences) flow cytometer equipped with 405 nm, 488 nm, and 633 nm lasers for excitation and 18 different emission channels. Forward and side scatter signals were detected and used to gate for single, and healthy cells. Fluorescence emission from three different channels (BV421, FITC, and BV510) were used to estimate fluorescence emission in the donor, acceptor, and FRET channels. Data was analyzed using custom MATLAB software.

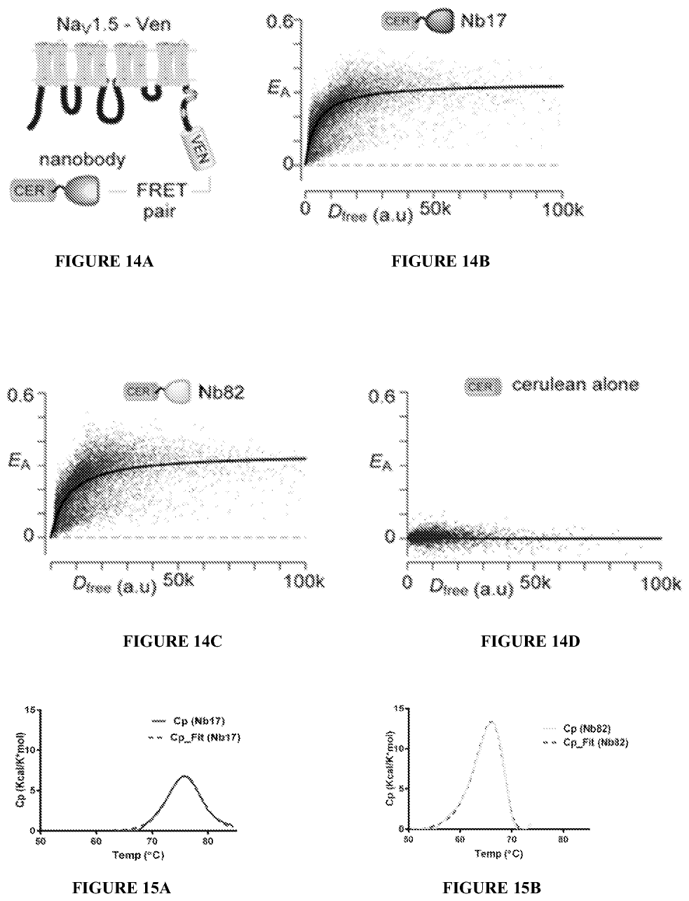

A cerulean fluorescent protein was attached to the carboxy-termini of both Nb17 and Nb82 (donor) and a versus fluorescent protein to the carboxy-terminus of Naᵥ1.5 (acceptor) (FIG. 14A), and fluorescence measurements were obtained using a flow cytometer. Stochastic expression of the FRET pairs resulted in variable FRET efficiencies (E_{A}) in individual cells. A saturating binding relation was then constructed by correlating E_{A} with the free donor concentration (D_{free}). Robust FRET was observed for both Nb17 (FIG. 14B, Figure Z) and Nb82 (FIG. 14C) with Naᵥ1.5 confirming baseline association of the heterologously-expressed nanobody in live cells. By contrast, co-expression of cerulean alone with Naᵥ1.5 did not show appreciable FRET (FIG. 14D). These findings demonstrate the robust interaction of Nb17 and Nb82 with Naᵥ1.5 in live cells, thus furnishing a new avenue to probe and manipulate Naᵥ channels in physiology. Nb17 and Nb82 show high affinity and are specific for Naᵥ1.4 and Naᵥ1.5 voltage-gated sodium channels.

A systematic analysis of nanobody interaction with the C terminal of the Naᵥ channels reveals that Nb17 binds (Naᵥ1.2/3/4/5/8/9). Each dot represents the FRET efficiency (E_{A}) calculated from an individual cell. Top, Naᵥ1.2 binds Nb17 with very weak affinity, middle Naᵥ1.4 binds strongly to Nb17. Bottom Naᵥ1.8 exhibits no binding to Nb17. The bar graph shows the relative association constant deduced from flow cytometric FRET 2-hybrid assay with 1-1 binding model (FIG. 18).

### Example 9

### Nb17 and Nb82 are Thermally Stable

The temperature stability of Nb17 and Nb82 were measured by differential scanning calorimetry (DSC). DSC is a thermal analysis technique in which the heat flow into or out of a sample is measured as a function of temperature or time, while the sample is exposed to a controlled temperature program. Nb17 is characterized by a T_{M} of 76° C. (FIG. 15A) and Nb82 by a T_{M} of 66° C. (FIG. 15B) suggesting a relatively stable protein suitable for functional and structural characterization. Notably, Nb17 undergoes reversible temperature denaturation whereas Nb82 undergoes irreversible denaturation. The different denaturation mechanisms probably originate from sequence differences. These different mechanisms are also reflected in the shape of the DSC curves (Rivas et al., Anal Biochem. 2021. 626:114240). Also, Nb17 has a van′t Hoff enthalpy of 118 kcal/mol that is close to what is expected for a protein of this size undergoing two-state unfolding. For Nb82 the irreversible transition is kinetically controlled, with a noticeable change in shape of the curve, and characterized by an activation energy of 86 kcal/mol.

### REFERENCES

[1] Fozzard, H. A., and Hanck, D. A. (1996) Structure and function of voltage-dependent sodium channels: comparison of brain II and cardiac isoforms, Physiol Rev 76, 887-926.
[2] Wulff, H., Christophersen, P., Colussi, P., Chandy, K. G., and Yarov-Yarovoy, V. (2019) Antibodies and venom peptides: new modalities for ion channels, Nat Rev Drug Discov 18, 339-357.
[3] Hutchings, C. J., Colussi, P., and Clark, T. G. (2019) Ion channels as therapeutic antibody targets, MAbs 11, 265-296.
[4] Che, T., English, J., Krumm, B. E., Kim, K., Pardon, E., Olsen, R. H. J., Wang, S., Zhang, S., Diberto, J. F., Sciaky, N., Carroll, F. I., Steyaert, J., Wacker, D., and Roth, B. L. (2020) Nanobody-enabled monitoring of kappa opioid receptor states, Nature communications 11, 1145.
[5] De Genst, E., Silence, K., Decanniere, K., Conrath, K., Loris, R., Kinne, J., Muyldermans, S., and Wyns, L. (2006) Molecular basis for the preferential cleft recognition by dromedary heavy-chain antibodies, Proc Natl Acad Sci USA 103, 4586-4591.
[6] Stortelers, C., Pinto-Espinoza, C., Van Hoorick, D., and Koch-Nolte, F. (2018) Modulating ion channel function with antibodies and nanobodies, Curr Opin Immunol 52, 18-26.
[7] Williams, W. A., Linley, J. E., Jones, C. A., Shibata, Y., Snijder, A., Button, J., Hatcher, J. P., Huang, L., Taddese, B., Thornton, P., Schofield, D. J., Thom, G., Popovic, B., Dosanjh, B., Wilkinson, T., Hughes, J., Dobson, C. L., Groves, M. A., Webster, C. I., Billinton, A., Vaughan, T. J., and Chessell, I. (2019) Antibodies binding the head domain of P2X4 inhibit channel function and reverse neuropathic pain, Pain 160, 1989-2003.
[8] Danquah, W., Meyer-Schwesinger, C., Rissiek, B., and Koch-Nolte, F. (2016) Nanobodies that block gating of the P2X7 ion channel ameliorate inflammation, Science translational medicine 8.
[9] Garza, J. A., Taylor, A. B., Sherwood, L. J., Hart, P. J., and Hayhurst, A. (2017) Unveiling a Drift Resistant Cryptotope within Marburgvirus Nucleoprotein Recognized by Llama Single-Domain Antibodies, Front Immunol 8, 1234.
[10] Duhoo, Y., Roche, J., Trinh, T. T. N., Desmyter, A., Gaubert, A., Kellenberger, C., Cambillau, C., Roussel, A., and Leone, P. (2017) Camelid nanobodies used as crystallization onent of the type IX secretion system from Porphyromonas gingivalis, Acta Crystallogr F Struct Biol Commun 73, 286-293.
[11] Herce, H. D., Deng, W., Helma, J., Leonhardt, H., and Cardoso, M. C. (2013) Visualization and targeted disruption of protein interactions in living cells, Nature communications 4, 2660.
[12] Ries, J., Kaplan, C., Platonova, E., Eghlidi, H., and Ewers, H. (2012) A simple, versatile method for GFP-based super-resolution microscopy via nanobodies, Nat Methods 9, 582-584.
[13] Buser, D. P., Schleicher, K. D., Prescianotto-Baschong, C., and Spiess, M. (2018) A versatile nanobody-based toolkit to analyze retrograde transport from the cell surface, Proc Natl Acad Sci USA 115, E6227-E6236.
[14] Senders, M. L., Hernot, S., Carlucci, G., van de Voort, J. C., Fay, F., Calcagno, C., Tang, J., Alaarg, A., Zhao, Y., Ishino, S., Palmisano, A., Boeykens, G., Meerwaldt, A. E., Sanchez-Gaytan, B. L., Baxter, S., Zendman, L., Lobatto, M. E., Karakatsanis, N. A., Robson, P. M., Broisat, A., Raes, G., Lewis, J. S., Tsimikas, S., Reiner, T., Fayad, Z. A., Devoogdt, N., Mulder, W. J. M., and Perez-Medina, C. (2019) Nanobody-Facilitated Multiparametric PET/MRI Phenotyping of Atherosclerosis, JACC Cardiovasc Imaging 12, 2015-2026.
[15] Wang, L., Zhang, L., Huang, B., Li, K., Hou, G., Zhao, Q., Wu, C., Nan, Y., Du, T., Mu, Y., Lan, J., Chen, H., and Zhou, E. M. (2019) A Nanobody Targeting Viral Nonstructural Protein 9 Inhibits Porcine Reproductive and Respiratory Syndrome Virus Replication, J Virol 93.
[16] Yu, C., Wang, L., Rowe, R. G., Han, A., Ji, W., McMahon, C., Baier, A. S., Huang, Y. C., Marion, W., Pearson, D. S., Kruse, A. C., Daley, G. Q., Wu, H., and Sliz, P. (2020) A nanobody targeting the LIN28:let-7 interaction fragment of TUT4 blocks uridylation of let-7, Proc Natl Acad Sci USA 117, 4653-4663.
[17] Caussinus, E., Kanca, O., and Affolter, M. (2011) Fluorescent fusion protein knockout mediated by anti-GFP nanobody, Nature structural & molecular biology 19, 117-121.
[18] Ruoff, K., Kilic, T., Devant, J., Koromyslova, A., Ringel, A., Hempelmann, A., Geiss, C., Graf, J., Haas, M., Roggenbach, I., and Hansman, G. (2019) Structural Basis of Nanobodies Targeting the Prototype Norovirus, J Virol 93.
[19] De Haard, H. J., Bezemer, S., Ledeboer, A. M., Muller, W. H., Boender, P. J., Moineau, S., Coppelmans, M. C., Verkleij, A. J., Frenken, L. G., and Verrips, C. T. (2005) Llama antibodies against a lactococcal protein located at the tip of the phage tail prevent phage infection, J Bacteriol 187, 4531-4541.
[20] Duarte, J. N., Cragnolini, J. J., Swee, L. K., Bilate, A. M., Bader, J., Ingram, J. R., Rashidfarrokhi, A., Fang, T., Schiepers, A., Hanke, L., and Ploegh, H. L. (2016) Generation of Immunity against Pathogens via Single-Domain Antibody-Antigen Constructs, J Immunol 197, 4838-4847.
[21] Scully, M., Cataland, S. R., Peyvandi, F., Coppo, P., Knobl, P., Kremer Hovinga, J. A., Metjian, A., de la Rubia, J., Pavenski, K., Callewaert, F., Biswas, D., De Winter, H., Zeldin, R. K., and Investigators, H. (2019) Caplacizumab Treatment for Acquired Thrombotic Thrombocytopenic Purpura, N Engl J Med 380, 335-346.
[22] Detalle, L., Stohr, T., Palomo, C., Piedra, P. A., Gilbert, B. E., Mas, V., Millar, A., Power, U. F., Stortelers, C., Allosery, K., Melero, J. A., and Depla, E. (2016) Generation and Characterization of ALX-0171, a Potent Novel Therapeutic Nanobody for the Treatment of Respiratory Syncytial Virus Infection, Antimicrob Agents Chemother 60, 6-13.
[23] Shilo, M., Motiei, M., Hana, P., and Popovtzer, R. (2014) Transport of nanoparticles through the blood-brain barrier for imaging and therapeutic applications, Nanoscale 6, 2146-2152.
[24] Yoder, J. B., Ben-Johny, M., Farinelli, F., Srinivasan, L., Shoemaker, S. R., Tomaselli, G. F., Gabelli, S. B., and Amzel, L. M. (2019) Ca(2+)-dependent regulation of sodium channels NaV1.4 and NaV1.5 is controlled by the post-IQ motif, Nature communications 10, 1514.
[25] Pardon, E., Laeremans, T., Triest, S., Rasmussen, S. G., Wohlkonig, A., Ruf, A., Muyldermans, S., Hol, W. G., Kobilka, B. K., and Steyaert, J. (2014) A general protocol for the generation of Nanobodies for structural biology, Nat Protoc 9, 674-693.
[26] Govaert, J., Pellis, M., Deschacht, N., Vincke, C., Conrath, K., Muyldermans, S., and Saerens, D. (2012) Dual beneficial effect of interloop disulfide bond for single domain antibody fragments, J Biol Chem 287, 1970-1979.
[27] Alzogaray, V., Urrutia, M., Berguer, P., Rossi, A., Zylberman, V., Pardo, R., Bonomi, H. R., and Goldbaum, F. A. (2019) Characterization of folding-sensitive nanobodies as tools to study the expression and quality of protein particle immunogens, J Biotechnol 293, 17-23.
[28] Ingram, J. R., Schmidt, F. I., and Ploegh, H. L. (2018) Exploiting Nanobodies' Singular Traits, Annu Rev Immunol 36, 695-715.
[29] Madeira, F., Park, Y. M., Lee, J., Buso, N., Gur, T., Madhusoodanan, N., Basutkar, P., Tivey, A. R. N., Potter, S. C., Finn, R. D., and Lopez, R. (2019) The EMBL-EBI search and sequence analysis tools APIs in 2019, Nucleic Acids Res 47, W636-W641.
[30] Winter, G., and McAuley, K. E. (2011) Automated data collection for macromolecular crystallography, Methods 55, 81-93.
[31] Kabsch, W. (2010) XDS: Integration, scaling, space-group assignment and post refinement, Acta Crystallogr D Biol Crystallogr 66, 125-132.
[32] McCoy, A. J., Grosse-Kunstleve, R. W., Adams, P. D., Winn, M. D., Storoni, L. C., and Read, R. J. (2007) Phaser crystallographic software, J Appl Crystallogr 40, 658-674.
[33] Emsley, P., Lohkamp, B., Scott, W. G., and Cowtan, K. (2010) Features and development of Coot, Acta Crystallogr D Biol Crystallogr 66, 486-501.
[34] Collaborative Computational Project, N. (1994) The CCP4 suite: programs for protein crystallography, Acta Crystallogr D Biol Crystallogr 50, 760-763.
[35] Berman, H., Henrick, K., and Nakamura, H. (2003) Announcing the worldwide Protein Data Bank, Nat Struct Biol 10, 980.

Although the invention has been described with reference to the above examples, it will be understood that modifications and variations are encompassed within the spirit and scope of the invention. Accordingly, the invention is limited only by the following claims.

1. A single-domain antibody (sdAb) that binds to voltage-gated sodium channel (Nav)1.4 or Nav1.5, wherein the sdAb comprises:
a) a complementarity-determining region (CDR) 1 having an amino acid sequence as set forth in SEQ ID NO:19, 22, 23, 26 or 29;
b) a CDR2 having an amino acid sequence as set forth in SEQ ID NO:20, 24, 27 or 30; and
c) a CDR3 having an amino acid sequence as set forth in SEQ ID NO:21, 25, 28 or 31.

2. The sdAb of claim 1, wherein the sdAb is selected from a camelid sdAb or a humanized sdAb.

3-4. (canceled)

5. The sdAb of claim 1, wherein the sdAb has a CDR1 having an amino acid sequence as set forth in SEQ ID NO:19, a CDR2 having an amino acid sequence as set forth in SEQ ID NO:20, and a CDR3 having an amino acid sequence as set forth in SEQ ID NO:21.

6. The sdAb of claim 1, wherein the sdAb has a CDR1 having an amino acid sequence as set forth in SEQ ID NO:23, a CDR2 having an amino acid sequence as set forth in SEQ ID NO:24, and a CDR3 having an amino acid sequence as set forth in SEQ ID NO:25.

7. The sdAb of claim 1, wherein the amino acid sequence of the sdAb is set forth in SEQ ID NO:5-SEQ ID NO:18.

8. The sdAb of claim 1, with the proviso that the sdAb does not bind to Nav1.7 or Nav1.9.

9. (canceled)

10. An isolated polynucleotide encoding the sdAb of claim 7.

11-12. (canceled)

13. An expression cassette comprising the polynucleotide of claim 10.

14-15. (canceled)

16. A vector comprising the expression cassette of claim 13.

17. A host cell comprising the polynucleotide of claim 10.

18. A pharmaceutical composition comprising the sdAb of claim 7 and a pharmaceutically acceptable carrier.

19. A method of detecting and/or capturing Nav1.4 or Nav1.5 in a sample comprising:
a) contacting the sample with the sdAb of claim 1; and
b) detecting and/or capturing a complex between the sdAb and the Nav1.4 or Nav1.5.

20.-23. (canceled)

24. A method of detecting a disease or condition in a subject comprising:
a) contacting a sample from the subject with the sdAb of claim 1; and
b) detecting the sdAb in the sample,
thereby detecting the disease or condition in the subject.

25. The method of claim 24, wherein the disease or condition is selected from the group consisting of cardiac arrhythmia, myotonia, neuropathic pain, hypokalemic periodic paralysis, Long QT syndrome, sudden cardiac death syndrome and Brugada syndrome.

26. The method of claim 24, wherein the disease or condition is selected from the group consisting of colon, prostate, breast, cervical, lung, pancreas, biliary, rectal, liver, kidney, testicular, brain, head and neck or ovarian cancer, melanoma, sarcoma, multiple myeloma, leukemia, and lymphoma.

27. A method of treating cardiac arrhythmia, myotonia or sudden cardiac death syndrome in a subject comprising administering to the subject a single-domain antibody (sdAb) that binds to voltage-gated sodium channel (Nav)1.4 or Nav1.5 for tissue-specific targeting of Nav1.4 or Nav1.5, thereby treating the treating cardiac arrhythmia, myotonia or sudden cardiac death syndrome.

28. The method of claim 27, wherein the sdAb has an amino acid sequence as set forth in SEQ ID NO:5 or 17.

29. A method of treating cancer in a subject comprising administering to the subject a single-domain antibody (sdAb) that binds to voltage-gated sodium channel (Nav)1.4 or Nav1.5 for tissue-specific targeting of Nav1.4 or Nav1.5, thereby treating the cancer.

30. (canceled)

31. The method of claim 29, wherein the sdAb has an amino acid sequence as set forth in SEQ ID NO:5 to SEQ ID NO:18.

32-34. (canceled)

35. The sdAb of claim 1, further comprising a polynucleotide encoding a cargo protein.

36-37. (canceled)
